SEQUENCE LISTING (9 sequences)

SEQ ID NO: 1 - protein, 1627 aa, Mus musculus
MPARTAPARVPALASPAGSLPDHVRRRLKDLERDGLTEKECVREKLNLLHEFLQTEIKSQLCDLETKLHKEELSEEGYLAKVKSLLNKDLSLENGTHTLTQKANGCPANGSRPTWRAEMADSNRSPRSRPKPRGPRRSKSDSDTLCKDTRHTAVETSPSSVATRRTTRQTTITAHFTKGPTKRKPKEESEEGNSAESAAEERDQDKKRRVVDTESGAAAAVEKLEEVTAGTQLGPEEPCEQEDDNRSLRRHTRELSLRRKSKEDPDREARPETHLDEDEDGKKDKRSSRPRSQPRDPAAKRRPKEAEPEQVAPETPEDRDEDEREEKRRKTTRKKLESHTVPVQSRSERKAAQSKSVIPKINSPKCPECGQHLDDPNLKYQQHPEDAVDEPQMLTSEKLSIYDSTSTWFDTYEDSPMHRFTSFSVYCSRGHLCPVDTGLIEKNVELYFSGCAKAIHDENPSMEGGINGKNLGPINQWWLSGFDGGEKVLIGFSTAFAEYILMEPSKEYEPIFGLMQEKIYISKIVVEFLQNNPDAVYEDLINKIETTVPPSTINVNRFTEDSLLRHAQFVVSQVESYDEAKDDDETPIFLSPCMRALIHLAGVSLGQRRATRRVMGATKEKDKAPTKATTTKLVYQIFDTFFSEQIEKYDKEDKENAMKRRRCGVCEVCQQPECGKCKACKDMVKFGGTGRSKQACLKRRCPNLAVKEADDDEEADDDVSEMPSPKKLHQGKKKKQNKDRISWLGQPMKIEENRTYYQKVSIDEEMLEVGDCVSVIPDDSSKPLYLARVTALWEDKNGQMMFHAHWFCAGTDTVLGATSDPLELFLVGECENMQLSYIHSKVKVIYKAPSENWAMEGGTDPETTLPGAEDGKTYFFQLWYNQEYARFESPPKTQPTEDNKHKFCLSCIRLAELRQKEMPKVLEQIEEVDGRVYCSSITKNGVVYRLGDSVYLPPEAFTFNIKVASPVKRPKKDPVNETLYPEHYRKYSDYIKGSNLDAPEPYRIGRIKEIHCGKKKGKVNEADIKLRLYKFYRPENTHRSYNGSYHTDINMLYWSDEEAVVNFSDVQGRCTVEYGEDLLESIQDYSQGGPDRFYFLEAYNSKTKNFEDPPNHARSPGNKGKGKGKGKGKGKHQVSEPKEPEAAIKLPKLRTLDVFSGCGGLSEGFHQAGISETLWAIEMWDPAAQAFRLNNPGTTVFTEDCNVLLKLVMAGEVTNSLGQRLPQKGDVEMLCGGPPCQGFSGMNRFNSRTYSKFKNSLVVSFLSYCDYYRPRFFLLENVRNFVSYRRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEIQNGASNSEIPYNGEPLSWFQRQLRGSHYQPILRDHICKDMSPLVAARMRHIPLFPGSDWRDLPNIQVRLGDGVIAHKLQYTFHDVKNGYSSTGALRGVCSCAEGKACDPESRQFSTLIPWCLPHTGNRHNHWAGLYGRLEWDGFFSTTVTNPEPMGKQGRVLHPEQHRVVSVRECARSQGFPDSYRFFGNILDRHRQVGNAVPPPLAKAIGLEIKLCLLSSARESASAAVKAKEEAATKD

SEQ ID NO: 2 - protein, 1632 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKRSRDPPASASQVTGIRAEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 3 - protein, 386 aa, Spiroplasma sp.
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 4 - DNA, 12 bp, synthetic construct
  modified_base 6: mod_base = OTHER; note = The residue at this position is 5-methylcytosine
  modified_base 10: mod_base = OTHER; note = The residue at this position is 5-hydroxymethylcytosine
  source 1..12: mol_type = other DNA
tcgatcgatcgt

SEQ ID NO: 5 - DNA, 12 bp, synthetic construct
  modified_base 2: mod_base = OTHER; note = The residue at this position is 5-hydroxymethylcytosine
  modified_base 6: mod_base = OTHER; note = The residue at this position is 5-methylcytosine
  source 1..12: mol_type = other DNA
acgatcgatcga

SEQ ID NO: 6 - DNA, 12 bp, synthetic construct
  source 1..12: mol_type = other DNA
acgatcgatcga

SEQ ID NO: 7 - DNA, 12 bp, synthetic construct
  modified_base 6: mod_base = OTHER; note = The residue at this position is 5-methylcytosine
  source 1..12: mol_type = other DNA
acgatcgatcga

SEQ ID NO: 8 - DNA, 12 bp, synthetic construct
  modified_base 6: mod_base = OTHER; note = The residue at this position is 5-methylcytosine
  modified_base 10: mod_base = OTHER; note = The residue at this position is 5-hydroxymethylcytosine
  source 1..12: mol_type = other DNA
ttgatcgatcgt

SEQ ID NO: 9 - DNA, 12 bp, synthetic construct
  source 1..12: mol_type = other DNA
ttgatcgatcgt

(19) United States
(12) Patent Application Publication
(10) Pub. No.: US 2025/0223640 A1
(43) Pub. Date: Jul. 10, 2025

(54) METHODS AND KITS FOR DETECTION OF METHYLATION STATUS

(71) Applicant: OSLO UNIVERSITETSSYKEHUS HF, Oslo (NO)

(72) Inventors: John Arne Dahl, Oslo (NO); Adam Brian Robertson, Oslo (NO); Arne Klungland, Oslo (NO); Linda Ellevog, Oslo (NO)

(21) Appl. No.: 19/095,303

(22) Filed: Mar. 31, 2025

Related U.S. Application Data
(63) Continuation of application No. 17/585,814, filed on Jan. 27, 2022.
Application No. 17/585,814 is a continuation of application No. 17/347,160, filed on Jun. 14, 2021, now abandoned.
Application No. 17/347,160 is a continuation of application No. 16/009,835, filed on Jun. 15, 2018, now Pat. No. 11,078,529.
Application No. 16/009,835 is a continuation of application No. 14/363,442, filed on Jun. 6, 2014, now Pat. No. 10,023,909.
(60) Provisional application No. 61/570,066, filed on Dec. 13, 2011.

(51) Int. Cl. C12Q 1/6869; C12Q 1/6844

(52) U.S. Cl. CPC C12Q 1/6869; C12Q 1/6846

(57) ABSTRACT

The present invention relates to methods and kits for the detection of 5-hydroxymethylcytosine (5hmC) and/or 5-methylcytosine (5mcC). In some embodiments, the present invention relates to methods and kits for detection of 5hmC and/or 5meC in nucleic acid (e.g., DNA, RNA). In some embodiments, the present invention relates to detection of 5hmC in genomic DNA, e.g., mammalian genomic DNA

### CROSS-REFERENCE TO RELATED APPLICATIONS

This application is a continuation of U.S. patent application Ser. No. 17/585,814, filed Jan. 27, 2022, which is a continuation of U.S. patent application Ser. No. 17/347,160, filed Jun. 14, 2021, which is a continuation of U.S. patent application Ser. No. 16/009,835, filed Jun. 15, 2018, allowed as U.S. Pat. No. 11,078,529, which is a continuation of U.S. patent application Ser. No. 14/363,442, filed Jun. 6, 2014, allowed as U.S. Pat. No. 10,023,909, which is a 371 U.S. National Phase Entry of International Patent Application No. PCT/US2012/069525, International Filing Date Dec. 13, 2012, which claims priority to expired U.S. Provisional Patent Application No. 61/570,066, filed Dec. 13, 2011, the contents of which are incorporated by reference in their entireties.

### INCORPORATION-BY-REFERENCE OF MATERIAL SUBMITTED ELECTRONICALLY

Incorporated by reference in its entirety herein is a nucleotide/amino acid sequence listing submitted concurrently herewith and identified as follows: One 16,511 bytes file named “INVEN_32320_306_SequenceListing.xml” created on Mar. 28, 2025.

### FIELD OF THE INVENTION

The present invention relates to methods and kits for the detection of 5-hydroxymethylcytosine (5hmC) and/or 5-methylcytosine (5meC). In some embodiments, the present invention relates to methods and kits for detection of 5hmC and/or 5meC in nucleic acid (e.g., DNA, RNA). In some embodiments, the present invention relates to detection of 5hmC in genomic DNA, e.g., mammalian genomic DNA.

### BACKGROUND OF THE INVENTION

The 5-hydroxymethylcytosine (5hmC) modification in mammalian DNA was discovered over 30 years ago1. At that time the 5hmC modification was suggested to be a rare and non-mutagenic DNA damage lesion2 and therefore it was given little attention. In early 2009 5hmC was identified again; however, in this year the importance of 5hmC in epigentics was realized as two independent groups began the initial characterization of the 5hmC modification. One group identified an enzyme capable of catalyzing the formation of 5hmC from 5-methylcytosine-Tet1³. The other group demonstrated that 5hmC was a stable modification present in specialized Purkinje neurons⁴. Further research has shown that Tet1, Tet2, and Tet3 are capable of catalyzing the oxidation of 5meC creating 5hmC⁵⁻⁷.

The molecular function of 5hmC remains poorly understood; however, it has been shown that 5hmC is involved in a variety of DNA transactions: it has been shown to be an intermediate in DNA demethylation^{3, 8}, to have a dual function in transcription⁹⁻¹¹ and in the case of aberrant 5hmC patterns to be involved in tumorigenesis⁷. While the function of the 5hmC modification remains unclear, it has become clear that identifying genomic regions that contain 5hmC will help to elucidate the function of this base. This need to identify genomic regions containing 5hmC has led to the development of suitable methods. Currently, there are several methods available to identify 5hmC; each method has certain limitations that are discussed below. The method described here allows for base specific resolution of (i) 5hmC and (ii) 5meC in DNA.

Currently, there are several methods that allow for the identification of 5hmC. These methods include antibodies raised against 5hmC^{9, 21, 22}, antibodies raised against cytosine 5-methylenesulfonane (CMS) the product of bisulfite treatment of 5hmC^{7, 23}, single molecule real time sequencing relying on DNA polymerase kinetics²⁴, restriction enzymes that are resistant or sensitive to 5hmC or β-glu-5hmC²⁵⁻²⁷ and three methods that take advantage of the β-glucosyltransferase: (i) incorporating a chemical tag into the substrate for the β-gt²⁸, (ii) the glucosylation, periodate oxidation, and biotinylation (GLIB) method²³, and (iii) the JBP1 pull-down assay targeting glu-5hmC¹²

The use of antibodies appears to be a reasonable choice to identify DNA modifications; however, we and others⁵ have seen that some of the currently available antibodies directed against 5hmC appear to be unable to sufficiently enrich for DNA that contains 5hmC; indeed one report demonstrates that one particular antisera raised against 5hmC is unable to differentiate 5hmC from 5meC⁵. It has been reported that antisera developed against 5hmC tends to prefer genomic regions dense in 5hmC content²². Moreover, the use of polyclonal antisera directed against 5hmC will provide an inherent problem, as there will be animal-to-animal variation in antigenic specificity to 5hmC that may affect the long-term usefulness of such antisera.

Upon treatment with sodium bisulfite 5hmC is converted to CMS, which after sequencing appears identical to bisulfite converted 5meC; therefore it has been shown that the use of bisulfite sequencing cannot distinguish between 5meC and 5hmC³⁰. Interestingly, one group has raised an antiserum directed against CMS^{7, 23}.

Single Molecule, Real Time (SMRT) sequencing takes advantage of the original Sanger sequencing technique; however, this method is able to distinguish between cytosine, 5meC, and 5hmC using the kinetic signature or speed that the polymerase passes over each base²⁴. This method, aside from being prohibitively expensive, requires a significant amount of DNA that is already enriched for 5hmC prior to use, which makes it dependent on a 5hmC enrichment assay. Because this method uses high-throughput sequencing it is cumbersome for the analysis of single or a few loci.

Several research groups and companies have identified restriction enzymes that are sensitive or resistant to 5hmC or β-glu-5hmC²⁵⁻²⁷. The principle behind these systems is that upon treatment with the restriction enzymes unmodified DNA is cleaved, resulting in reduced signal in a qPCR reaction. This reduction in signal is then compared to an undigested sample and the difference in qPCR signals is proportional to the amount of 5hmC present in the initial sample. These methods work quite well for genomic regions that contain significant amounts of 5hmC; however, because the restriction sites recognized by these enzymes are 4-6 bp in length these restriction endonuclease based methods can, at best, only recognize 1/16 of all 5hmC modifications.

Three groups have developed methods that take advantage of the specificity that the β-gt has for 5hmC. The first group²⁸ incorporated an azide group into the substrate for the β-gt—UDP-glucose—creating UDP-6-N₃-Glucose. After the azide modified glucose was incorporated into 5hmC containing DNA by the β-gt, a second group could be added to the 6-N₃-glu-5hmC using “click” chemistry. This second chemical group could contain a biotin for pull down, a fluorescent probe for quantification, and theoretically any group that could be coupled to the modified glucose using “click” chemistry. The primary drawback to this method is that UDP-6-N₃-glucose is not commercially produced and requires significant expertise in organic chemistry to synthesize. Additionally, this targeting strategy of 5hmC has been combined with a primer extension assay and shown to allow for base specific resolution as a chemical group can be linked to 6-N₃-glu-5hmC that blocks a DNA polymerase. By blocking the polymerase the terminal base can be assumed to have originally contained a 5hmC modification. The use of this method for base specific resolution has substantial problems as every end that terminates in a C must be assumed to be a 5hmC. While this effect can potentially be averaged with several high throughput sequencing reads assuming highly optimized enzyme to DNA ratios, it remains problematical for single gene analysis.

A second approach using the β-gt to identify genomic regions uses the glucosylation, periodate oxidation, biotinylation (GLIB) method²³. In this method after the transfer of glucose to 5hmC, the resulting β-glu-5hmC is oxidized using NaIO₄ which creates reactive aldehydes on the glucose moiety attached to 5hmC. These oxidized glucose molecules can then be reacted with commercially available aldehyde reactive probes containing a biotin modification. This biotinylation allows for the efficient pull down of 5hmC containing DNA.

Finally, the third approach utilizing the β-gt for the identification of 5hmC involves the specific recognition of this modified base by a second protein—J-base binding protein or JBP1. Because the only difference between β-glucosyl-5hmC and the J-base is an amino group, it was reasoned that JBP1 may be able to specifically interact with β-glu-5hmC. JBP1 was indeed able to specifically interact with β-glu-5hmC¹². Therefore, when JBP1 was covalently linked to epoxy modified magnetic beads it allowed for the pull down of the β-glu-5hmC containing DNA. After removing protein from the pulled down DNA it was demonstrated by gene specific qPCR that it was possible to enrich for DNA containing 5hmC¹². Mechanistically, this method provides two degrees of specificity for the identification of 5hmC in genomic DNA: first, the β-gt can only modify cytosines in DNA that are hydroxymethylated and second, JBP1 interacts specifically with β-glu-5hmC. Like all DNA pull down methods the very optimal resolution of this method can identify a 5hmC base within about 50-100 base pairs; this limitation is due to the inability to reliably identify DNA fragments of a shorter length using currently available molecular biology methods. Another consideration when using this protocol is that this method may over-represent DNA regions that contain high levels of 5hmC. This potential over-representation could possibly occur because in 5hmC dense regions more JBP1 can interact with the DNA and pull down these regions more efficiently.

Improved methods for detecting 5-hydroxymethylcytosine residues in DNA are needed. In particular, methods that can discriminate between 5meC and 5hmC are needed, as well as methods which can identify 5meC and 5hmC at single base resolution.

### SUMMARY OF THE INVENTION

The present invention relates to methods and kits for the detection of 5-hydroxymethylcytosine (5hmC) and/or 5-methylcytosine (5meC). In some embodiments, the present invention relates to methods and kits for detection of 5hmC and/or 5meC in nucleic acid (e.g., DNA, RNA). In some embodiments, the present invention relates to detection of 5hmC in genomic DNA, e.g., mammalian genomic DNA.

In some embodiments, the present invention provides processes for detecting 5-methylated and/or other modified cytosine residues in a nucleic acid sample comprising: replicating said nucleic acid sample under conditions such that 5-methylated cytosine residues are maintained and said other modified cytosine residues are diluted; treating said replicated nucleic acid sample to convert unmodified cytosine residues to a uracil or thymidine residues; and reading the sequence of said replicated nucleic acid sample wherein 5-hydroxymethylated cytosine residues are identified as residues that are read by sequencing as a thymidine or uracil residue in said replicated nucleic acid sample. In some embodiments, the nucleic acid sample is divided into at least first and second portions and said replicating and treating steps are performed on said first portion, and comparing the sequence of said first nucleic acid portion with the sequence of said second nucleic acid portion, wherein said other modified cytosine residues are identified as residues that are read by sequencing as a uracil or thymidine residue in said first nucleic acid portion and as a cytosine residue at the corresponding position in said second nucleic acid portion and wherein 5-methylated cytosine residues are identified as residues that are read as cytosine residues in both of said first and second nucleic acid portions. In some embodiments, the replication of said first portion further comprises: a) replicating said nucleic acid with a tagged primer to provide tagged replicated nucleic acid; b) treating said tagged replicated nucleic acid strands with a DNA methyltransferase to provide tagged 5-methylcytosine-modified replicated nucleic acid; c) isolating said tagged 5-methylcytosine-modified replicated nucleic acid; d) treating said isolated tagged 5-methylcytosine-modified replicated nucleic acid with bisulfite to convert unmodified cytosine residues to uracil residues; and e) replicating said isolated tagged bisulfite-treated nucleic acid with a polymerase to provide a first bisulfite treated nucleic acid portion. In some embodiments, the tagged primer is a biotinylated primer. In some embodiments, the other modified cytosine residues are selected from the group consisting of 5-hydroxymethyl cytosine, beta-glu-5-hydroxymethyl cytosine, alpha-glucosyl-5-hydroxymethylcytosine, beta-glucopyranosyl-alpha-glycosyl-5-hydroxymethylcytosine (gentiobiosyl-5-hydroxymethylcytosine), 5-formylcytosine and 5-carboxycytosine.

In some embodiments, the replicating said first portion under conditions such that 5-methylated cytosine residues are maintained and 5-hydroxymethylated cytosine residues are diluted comprises replicating said nucleic acid with a polymerase to provide replicated nucleic acid and treating said replicated nucleic acid with an enzyme to 5-methylate cytosine residues. In some embodiments, the steps of replication and treating with an enzyme are performed one or more times. In some embodiments, the steps of replication and treating with an enzyme are repeated 5 or more times. In some embodiments, the steps of replication and treating with an enzyme are repeated 7 or more times. In some embodiments, the steps of replication and treating with an enzyme are repeated 10 or more times. In some embodiments, the steps of replication and treating with an enzyme are performed from about 1 to about 20 times or more. In some embodiments, replication is by a polymerase chain reaction. In some embodiments, replication is by a primer extension reaction. In some embodiments, the enzyme is a DNA methyltransferase. In some embodiments, the DNA methyltransferase is DNMT1. In some embodiments, the DNA methyltransferase is M.Sss1.

In some embodiments, the treating said first and second portions to convert unmodified cytosine residues to thymidine residues further comprises treating said first and second nucleic acid portions with bisulfite to convert unmodified cytosine residues to uracil resides and replicating said first and second nucleic acid portions with a polymerase to convert said uracil residues into thymidine residues. In some embodiments, replication is performed 1 or more times. In some embodiments, replication is performed 5 or more times. In some embodiments, replication is performed 7 or more times. In some embodiments, replication is performed 10 or more times. In some embodiments, replication is repeated from about 1 to about 20 times. In some embodiments, the replication is by a polymerase chain reaction. In some embodiments, the replication is by a primer extension reaction.

In some embodiments, the nucleic acid sample is selected from the group consisting of human, plant, mouse, rabbit, hamster, primate, fish, bird, cow, sheep, pig, viral, bacterial and fungal nucleic acid samples.

In some embodiments, the processes further comprise comparing the presence of 5-hydroxymethylcytosine and/or 5-methylcytosine in said nucleic acid in said sample to a reference standard, wherein an increased or decreased level of 5-hydroxymethylcytosine and/or 5-methylcytosine in said nucleic acid is indicative of the presence of a disease or of the probable course of a disease. In some embodiments, the processes further comprise the step of providing a diagnoses or prognoses based on an increased or decreased level of 5-hydroxymethylcytosine and/or 5-methylcytosine in said nucleic acid as compared to a reference standard. In some embodiments, the disease is cancer. In some embodiments, the nucleic acid sample is genomic DNA.

In some embodiments, the present invention provides processes for detecting methylated and hydroxymethylated cytosine residues in a nucleic acid sample comprising: a) dividing said sample into at least first and second untreated portions; b) replicating said first portion with a tagged primer and a polymerase to provide parent and tagged replicated nucleic acid; c) treating said parent and said tagged replicated nucleic acid strands with a DNA methyltransferase to provide tagged 5-methylcytosine-modified replicated nucleic acid; d) isolating said tagged 5-methylcytosine-modified replicated nucleic acid; e) treating said isolated tagged 5-methylcytosine-modified replicated nucleic acid with bisulfite to convert unmodified cytosine residues to uracil residues; f) replicating said isolated tagged bisulfite-treated nucleic acid with a polymerase to provide a first bisulfite treated nucleic acid portion; g) sequencing said first bisulfite treated nucleic acid portion; h) treating said second portion with bisulfite to convert unmodified cytosine residues to uracil residues; i) replicating said bisulfite-treated nucleic acid with a polymerase to provide a second bisulfite treated nucleic acid portion; j) sequencing said second bisulfite treated nucleic acid portion; and k) comparing the sequence of said first bisulfite treated nucleic acid portion with the sequence of said second bisulfite treated portion, wherein 5-hydroxymethylated cytosine residues are identified as residues that are read by sequencing as a uracil or thymidine residue in said first bisulfite treated nucleic acid portion and as a cytosine residue at the corresponding position in said second bisulfite treated nucleic acid portion and wherein 5-methylated cytosine residues are identified as residues that are read as cytosine residues in said first and second bisulfite treated portions. In some embodiments, said second portion is replicated with a polymerase prior to said sequencing step. In some embodiments, said steps b, c and d are repeated from about 2 to about 20 times. In some embodiments, said steps e and h are repeated from about 2 to about 20 times. In some embodiments, said replicating in steps b, e and h is by polymerase chain reaction. In some embodiments, the processes further comprise comparing the presence of 5-hydroxymethylcytosine and/or 5-methylcytosine in said nucleic acid in said sample to a reference standard, wherein an increased or decreased level of 5-hydroxymethylcytosine and/or 5-methylcytosine in said nucleic acid is indicative of the presence of a disease or of the probable course of a disease. In some embodiments, the processes further comprise the step of providing a diagnoses or prognoses based on an increased or decreased level of 5-hydroxymethylcytosine and/or 5-methylcytosine in said nucleic acid as compared to a reference standard. In some embodiments, the disease is cancer. In some embodiments, the nucleic acid sample is genomic DNA.

In some embodiments, the present invention provides a process for predicting a predisposition to as disease in a subject, diagnosing a disease in a subject, predicting the likelihood of recurrence of disease in a subject, providing a prognosis for a subject with a disease, or selecting a subject with a disease for treatment with a particular therapy, comprising: a) providing a genomic DNA sample from said subject; and b) detecting the methylation status of predetermined portions of said genomic DNA sample by the processes described above,

wherein an altered level of 5-hydroxymethylcytosine and/or 5-methylcytosine methylation of said predetermined portions of said genomic DNA to a reference methylation status provides an indication selected from the group consisting of an indication of a predisposition of the subject to a disease, an indication that the subject has a disease, an indication of the likelihood of recurrence of a disease in the subject, an indication of survival of the subject, and an indication that the subject is a candidate for treatment with a particular therapy. In some embodiments, the disease is a cancer. In some embodiments, the subject is a human.

In some embodiments, the present invention provides a kit for determination of the methylation status of a nucleic acid sample comprising: 1) container(s) with reagents for methylating nucleic acid; and 2) container(s) with reagents for bisulfite sequencing. In some embodiments, the kits further comprise nucleic acid primers for amplifying and/or sequencing a region of said nucleic acid sample. In some embodiments, the kits further comprise a computer readable medium comprising a computer algorithm that analyzes sequence data obtained using said kit.

Additional embodiments will be apparent to persons skilled in the relevant art based on the teachings contained herein.

### DESCRIPTION OF THE DRAWINGS

FIG. 1. Schematic depiction of certain embodiments of the present invention, which applies bisulfite conversion and sequencing of “A” untreated DNA which will be used as a reference as it will detect the total of both 5meC and 5hmC. The method involves a 5hmC dilution assay, diluting 5hmC in the total pool of DNA fragments while maintaining 5meC. This dilution is achieved through sequential rounds of one cycle of PCR amplification (dilution) and treatment of the DNA with the DNA maintenance methyltransferase DNMT1 which enzymatically and specifically maintains 5meC by adding a methyl group uniquely to the unmethylated strand of the hemimethylated PCR products (this sample is referred to as “B” in FIG. 1). After a few rounds of this assay we apply bisulfite conversion and sequencing of the treated DNA sample, B. Bases that read as cytosine from this sample must have been protected against bisulfite conversion because of 5meC and not 5hmC. By comparing “B” to the reference sample “A” we can easily detect all base positions containing 5hmC.

FIG. 2. Bisulfite conversion of DNA results in conversion of unmodified cytosine (C) to uracil (U) that will be read as thymine (T) upon sequencing of PCR amplified DNA. Both 5meC and 5hmC are protected against conversion and will not be converted to U. Therefore, both bases will be read as C upon sequencing. Bisulfite conversion is a well established technology that has long been regarded as the gold standard for detection of 5meC, and it was not until recently (2010) that it was reported in the scientific literature that bisulfite conversion can not distinguish between 5meC and 5hmC.

FIG. 3A-C. Mouse DNMT1, human DNMT1 and M. SssI preferentially methylate hemi-5meC DNA. 100 ng of each DNA substrate was incubated with 2 units mouse DNMT1, human DNMT1, or SssI methyltransferase as described in “materials and methods” section.

FIG. 4A-C. Validation of the feasibility of the 5hmC dilution assay. (A) The double stranded DNA oligo used in the validation contains three CpG sites where one is hemi for 5meC, a second one is having no modification and a third one is hemi for 5hmC. (B) Bisulfite conversion and sequencing of the unmodified bottom strand of the oligo in (A), when the oligo has not been subject to DNMT1 treatment, showed that all Cs were converted and read as T. (100% T is equal to 16 out of 16 individual clones being read as T at the C position of the CpG site). (C) Treatment with DNMT1 prior to bisulfite conversion and sequencing resulted in the addition of a methyl group to the unmethylated C of the CpG site hemi for 5meC in 87.5% of the oligoes. (Sequencing read a C at the C position of the CpG site in 14 out of 16 clones). No addition of a methyl group was observed across from C or 5hmC.

FIG. 5A-B. Schematic presentation of the method for distinct identification of 5hmC and 5meC at base specific resolution. (A) A scheme following the C bases of the CpG sites of a dsDNA oligo which contains three CpG sites where one is having 5meC at both strands, a second one is having no modification and a third one is having 5hmC at both strands. The CpG sites are followed through one round of PCR (melting, primer annealing and elongation) and DNMT1 treatment before visualization of bisulfite treatment and PCR (30 cycles) which generates the bases that will be read in the sequencing. (B) Flow chart of the experimental procedure involved in the 5hmC dilution assay.

FIG. 6A-B. Preferential maintenance of 5meC over 5hmC. The double stranded DNA oligo used here contains three CpG sites, one having 5meC at both strands, a second one having no modification and a third one having 5hmC at both strands. (A) Bisulfite conversion and sequencing of the untreated oligo showed that only modified Cs were protected from being converted (100% for both 5meC and 5hmC), whereas unmodified cytosines were all converted. (B) Taking the double stranded DNA oligo through three rounds of the dilution assay, involving PCR and treatment with DNMT1, prior to bisulfite conversion and sequencing resulted in preferential maintenance of 5meC over 5hmC. There was no methylation across from 5hmC in any of the three rounds as the initial 5hmC modified strands made up only 9% of the total pool after three rounds of the dilution assay. (One would expect 50% after one round, 25% after two rounds and 12.5% after three rounds when there is no maintenance at all). The 5meC base was preferentially maintained, thus resulted in a higher number of Cs protected in the bisulfite conversion and a significantly higher read out than the 5hmC base. No addition of a methyl group was observed across from either C or 5hmC.

FIG. 7A-B. Schematic presentation of the method for distinct identification of 5hmC and 5meC at base specific resolution making use of strand specific assessment. (A) A scheme following the C bases of the CpG sites of a dsDNA oligo which contains three CpG sites where one is having 5meC at both strands, a second one is having no modification and a third one is having 5hmC at both strands. The CpG sites are followed through one round of strand specific primer extension PCR (melting, primer annealing and elongation) and DNMT1 treatment. The primer used may contain a biotin tag, or other tag, to allow for selection/isolation of the newly synthesized strand. The newly synthesized strand undergoes bisulfite treatment and PCR (30 cycles or other number) which generates the bases that will be read in the sequencing. (B) Flow chart of the experimental procedure involved in the 5hmC dilution/loss assay applying primer extension and strand specific assessment. p FIG. 8. Amino acid sequence for DNMT1 (Mus musculus) Recombinant Accession Number: GenBank: AAH53047.1 (SEQ ID NO:1).

FIG. 9. Amino acid sequence for DNMT1 (Homo sapiens) Accession Number: GenBank: AAI44094.1 (SEQ ID NO:2).

FIG. 10. Amino acid sequence for M.Sss1 (Spiroplasma sp. (strain MQ1)) site-specific DNA-methyltransferase (SEQ ID NO:3).

FIG. 11. Schematic depiction of of a 5hmC loss assay of the present invention utilizing biotinylated primers and streptavidin capture beads. Right panel, top, shows representative sequencing results of 10 clones for the conventional bisulfite assay, referred to as A, where both 5meC and 5hmC will be read as cytosine after treatment, and sequencing results of 10 clones for the methyl transfer assay/5hmC loss assay, referred to as B, where only 5meC will be read as cytosine after treatment. Cytosines in a CG sequence context (CpG) protected from bisulfite conversion are illustrated as filled circles whereas cytosines in a CG sequence context which undergo deamination to Uracil in the bisulfite treatment are illustrated as open circles. The combination of the standard bisulfite assay data, A, where both 5meC and 5hmC will be read as cytosine after treatment and the methyl transfer assay, B, where only 5meC will be read as a cytosine after treatment allows for determination of position and quantity of 5hmC, from the simple calculation: A−B=5hmC. This quantification is outlined in the bottom of the right panel. These experimental results have been reproduced in 15 independent experiments.

FIG. 12. Schematic and graphs showing identification of two 5hmC containing islands CpGs, that is 5hmC in a CG sequence, in the TRIM31 gene in human brain DNA using the assay depicted in FIG. 11. Positions of the CpGs are schematically depicted (not to scale) and the quantity of 5hmC and 5meC at those to cytosine positions are given in the bar graphs.

FIG. 13. Bar graph showing the results of an experiments where methyl transferase is blocked by addition of a chemical group to 5hmC.

### DEFINITIONS

To facilitate an understanding of the present invention, a number of terms and phrases are defined below:

As used herein, the term “sensitivity” is defined as a statistical measure of performance of an assay (e.g., method, test), calculated by dividing the number of true positives by the sum of the true positives and the false negatives.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (e.g., epigenetic marker; e.g., 5hmC at one or more particular locations) in a positive sample.

As used herein, the term “dilution” refers to the reduction of non-5-methyl modified cytosine residues (e.g., 5-hydroxymethl cytosine residues) in a nucleic acid sample as compared to the 5-methyl cytosine residues through repeated rounds of replication of said DNA sample.

As used herein the term “non-5-methyl cytosine modified cytosine residues” refers to modified cytosine residues other than 5-methyl cytosine, for example, 5-hydroxymethyl cytosine, b-glu-5-hydroxymethyl cytosine, 5-formyl-cytosine and 5-carboxycytosine.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like). Various parameters and definitions for CpG islands exist; for example, in some embodiments, CpG islands are defined as having a GC percentage that is greater than 50% and with an observed/expected CpG ratio that is greater than 60% (Gardiner-Garden et al. (1987) J Mol. Biol. 196:261-282; Baylin et al. (2006) Nat. Rev. Cancer 6:107-116; Irizarry et al. (2009) Nat. Genetics 41:178-186; each herein incorporated by reference in its entirety). In some embodiments, CpG islands may have a GC content >55% and observed CpG/expected CpG of 0.65 (Takai et al. (2007) PNAS 99:3740-3745; herein incorporated by reference in its entirety). Various parameters also exist regarding the length of CpG islands. As used herein, CpG islands may be less than 100 bp; 100-200 bp, 200-300 bp, 300-500 bp, 500-750 bp; 750-1000 bp; 1000 or more bp in length. In some embodiments, CpG islands show altered methylation patterns (e.g., altered 5hmC patterns) relative to controls (e.g., altered 5hmC methylation in cancer subjects relative to subjects without cancer; tissue-specific altered 5hmC patterns; altered 5hmC patterns in biological samples from subjects with a neoplasia or tumor relative to subjects without a neoplasia or tumor. In some embodiments, altered methylation involves increased incidence of 5hmC. In some embodiments, altered methylation involves decreased incidence of 5hmC.

As used herein, the term “CpG shore” or “CpG island shore” refers to a genomic region external to a CpG island that is or that has potential to have altered methylation (e.g., 5hmC) patterns (see, e.g., Irizarry et al. (2009) Nat. Genetics 41:178-186; herein incorporated by reference in its entirety). CpG island shores may show altered methylation (e.g., 5hmC) patterns relative to controls (e.g., altered 5hmC in cancer subjects relative to subjects without cancer; tissue-specific altered 5hmC patterns; altered 5hmC in biological samples from subjects with neoplasia or tumor relative to subjects without neoplasia or tumor. In some embodiments, altered methylation involves increased incidence of 5hmC. In some embodiments, altered methylation involves decreased incidence of 5hmC. CpG island shores may be located in various regions relative to CpG islands (see, e.g., Irizarry et al. (2009) Nat. Genetics 41;178-186; herein incorporated by reference in its entirety). Accordingly, in some embodiments, CpG island shores are located less than 100 bp; 100-250 bp; 250-500 bp; 500-1000 bp; 1000-1500 bp; 1500-2000 bp; 2000-3000 bp; 3000 bp or more away from a CpG island.

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body.

As used herein, “an individual is suspected of being susceptible to metastasized cancer” is meant to refer to an individual who is at an above-average risk of developing metastasized cancer. Examples of individuals at a particular risk of developing cancer of a particular type (e.g., colorectal cancer, bladder cancer, breast cancer, prostate cancer) are those whose family medical history indicates above average incidence of such cancer type among family members and/or those who have already developed cancer and have been effectively treated who therefore face a risk of relapse and recurrence. Other factors which may contribute to an above-average risk of developing metastasized cancer which would thereby lead to the classification of an individual as being suspected of being susceptible to metastasized cancer may be based upon an individual's specific genetic, medical and/or behavioral background and characteristics.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specfic PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, 5-hydroxymethylcytosine, b-glucosyl-5-hydroxymethylcytosine, 5-formylcytosine, and 5-carboxycytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) processed transcript. The mRNA functions during translation to specify the sequence or order of amino acids in a nascent polypeptide.

### DETAILED DESCRIPTION OF THE INVENTION

The present invention relates to methods and kits for the detection of 5-hydroxymethylcytosine (5hmC) and/or 5-methylcytosine (5meC). In some embodiments, the present invention relates to methods and kits for detection of 5hmC and/or 5meC in nucleic acid (e.g., DNA, RNA). In some embodiments, the present invention relates to detection of 5hmC in genomic DNA, e.g., mammalian genomic DNA. Current methods available for identifying 5hmC have a resolution limit of about 50-200 base pairs. Many of the current methods are limited by the step of bisulfite conversion which cannot distinguish between 5-methylcytosine (5meC) and 5hmC. The present invention addresses both of these problems. First, the present invention allows for discrimination between 5meC and 5hmC DNA modifications. Second, the present invention allows for the detection of both 5meC and 5hmC at single base resolution.

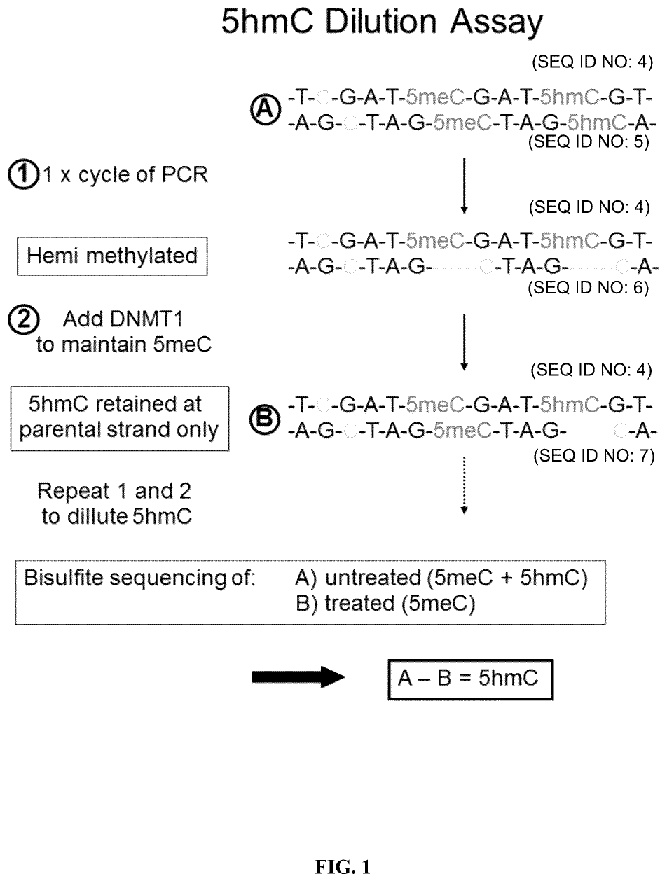

The method described here identifies 5-hydroxymethylcytosine (5hmC) in DNA with single base resolution. Additionally, this method can identify 5meC at base specific resolution concurrently with 5hmC. The method employed takes advantage of the fact that the DNMT1 methyltransferase cannot methylate across from a 5hmC (or modified 5hmC; as is the case for β-glucosyl-5-hydroxymethylcytosine) and preferentially methylates across from 5-methylcytosine (5meC). After sequential rounds of one cycle of PCR amplification and treatment of the DNA with DNMT1 the population of DNAs containing 5hmC is diluted by a factor of two whereas the population containing 5meC remains stable. This dilution coupled with bisulfite conversion allows for the base specific identification of DNA residues that contain 5hmC (FIG. 1).

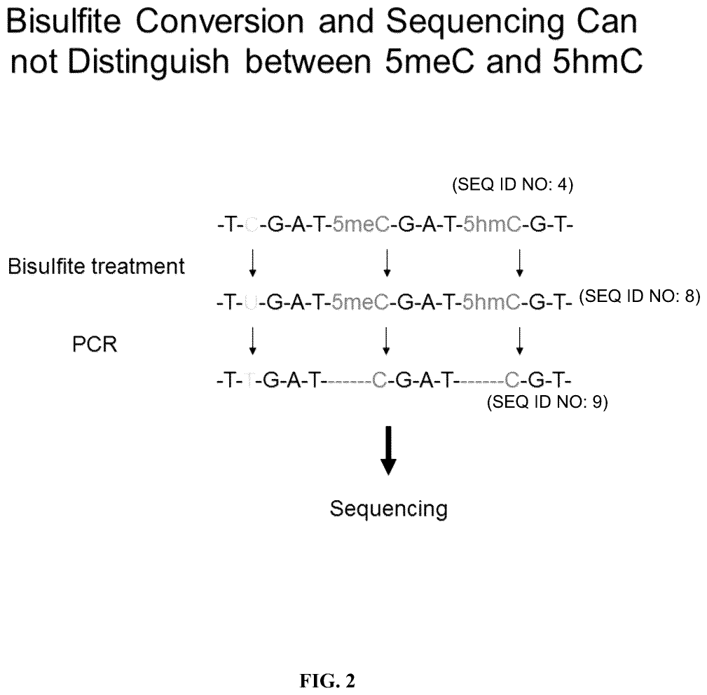

Bisulfite conversion of DNA results in conversion of unmodified cytosine (C) to uracil (U) that will be read as thymine (T) upon sequencing of PCR amplified DNA. Both 5meC and 5hmC are protected against conversion and will not be converted to U. Therefore they will both be read as C upon sequencing (see FIG. 2). Bisulfite conversion is a well established technology that has long been regarded as the gold standard for detection of 5meC, and it was not until recently (2010) that it was reported in the scientific literature that it cannot distinguish between 5meC and 5hmC³⁰. However, the method described here takes advantage of this fact to create a reference data set (referred to as “A” in FIG. 1).

In preferred embodiments of the present invention, 5hmC is diluted in the total pool of DNA while maintaining 5meC. This dilution is achieved through sequential rounds of one cycle of PCR amplification and treatment of the DNA with the DNA maintenance methyltransferase DNMT1 which enzymatically and specifically maintains 5meC only by adding a methyl group to the unmethylated strand of the hemimethylated PCR products (this sample is referred to as “B” in FIG. 1). After one or more rounds of this assay, bisulfite conversion is performed followed by sequencing of the treated DNA sample, where 5meC now is the predominant modification. It is contemplated that all or most bases read as C from this sample must have been protected against conversion because of 5meC and not 5hmC. By comparing to the reference sample “A” it is possible to detect all base positions containing 5hmC. The dilution may be achieved on a genome wide basis or with respect to a particular gene locus or portion of a gene. In preferred embodiments, the region of dilution is defined by primers utilized for replication and/or amplification of a target region of interest.

Accordingly, in some embodiments, the present invention provides processes for detecting or determining the 5meC and/or 5hmC status of a nucleic acid sample, and in particularly preferred embodiments, the 5meC and/or 5hmC status or a predetermined region of a genomic DNA sample. In some preferred embodiments, the predetermined region (or target region of interest) corresponds to a gene locus of interest, or to a portion of a gene. In some embodiments, the predetermined region is defined by nucleic acid primers utilized for replication or amplification of the predetermined region.

In some preferred embodiments, the nucleic acid sample is divided into at least two portions for further analysis. In some embodiments, the first portion is replicated under conditions such that 5-methylated cytosine residues are maintained and 5-hydroxymethylated cytosine residues are diluted. The present invention is not limited to any particular level of dilution. For example, the 5-hydroxymethylated cytosine residues may be diluted by a factor of 1.5, 2, 5, 10, 20, 40, 100, 200, 400, 800, 1600 or more.

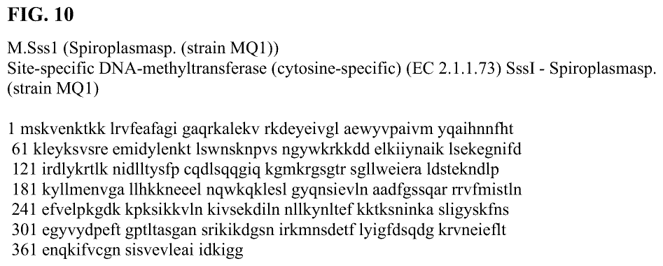

In some embodiments, the dilution of 5-hydroxymethylated cytosine residues is accomplished by replicating the nucleic acid (preferably replicating the predetermined region) with a polymerase to provide replicated nucleic acid and then treating the replicated nucleic acid with an enzyme that adds a methyl group to the unmethylated strand of the hemimethylated nucleic acid, but that does not add a hydroxymethyl group to the unhydroxymethylated strand of hemihydroxymethylated nucleic acid. The present invention is not limited to the use of any particular enzyme. In some embodiments, the enzyme is an enzyme that maintains the DNA methylation status of a nucleic acid, for example a DNA methyltransferase (DNMT). Example of DNA methyltransferases include, but are not limited to, mouse DNMT1 (SEQ ID NO:1; FIG. 8), human DNMT1 (SEQ ID NO:9, FIG. 8) or M.SssI (Spiroplasma sp.) DNMT (SEQ ID NO:3, FIG. 10), or a homolog or variant thereof. In some embodiments, the homologs or variants have the activity of adding a methyl group to the unmethylated strand of a hemimethylated nucleic acid. In some embodiments, the homologs or variants have at least 50%, 60%, 70%, 80%, 90%, 95%, or 99% identity to SEQ ID NOs:1, 2 or 3 and/or have the activity of adding a methyl group to the unmethylated strand of a hemimethylated nucleic acid.

In some embodiments, the replication step is performed via one or more rounds of polymerase chain reaction. In preferred embodiments, a predetermined region is replicated by extension from nucleic acid primers defining the 5′ and 3′ boundaries of the predetermined region. The replicated nucleic acid is then treated with a DNA methylation enzyme as described above to maintain 5-methylcytosine methylation of the predetermined region and then the process is repeated until a desired level of dilution of 5-hydroxymethylated cytosine residues as compared to 5-methylated residues is achieved. In some embodiments, the level of dilution per cycle is preferably about 2 fold, but maybe as low as 1.1. In some embodiments, the level of maintenance of 5-methyl cytosine residues is about 100%, but may be as low as 10% and still provide effective determination of and discrimination between 5meC and 5hmC residues in the predetermined region. In some embodiments, the number of cycles of replication and treatment with DNA methylation enzyme may 1, 2, 3, 5, 7, 10 or 20 cycles or more, or between about 1 and 20 cycles.

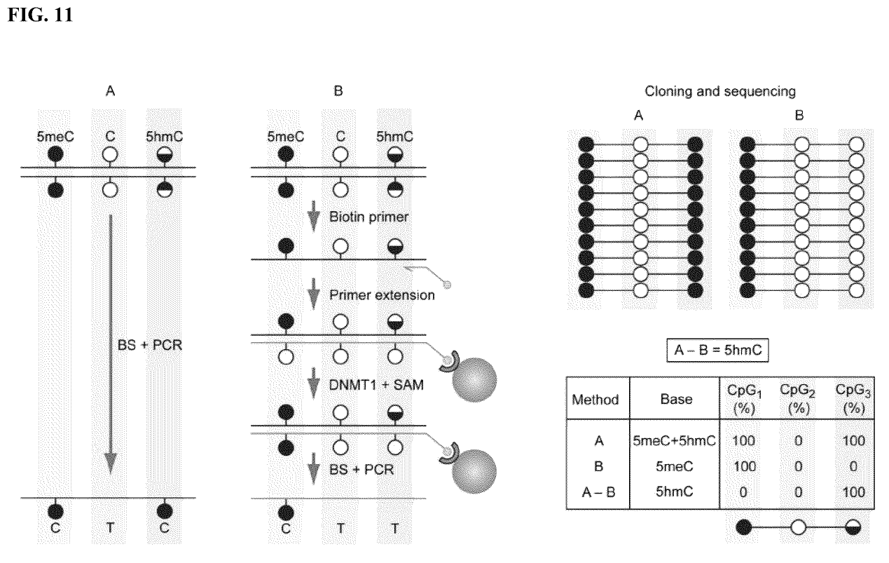

In some embodiments, tagged primers are used in the replication step so that tagged extension products from the replication step may be isolated using a tag binding reagent and used in subsequent steps, such as for treatment with a DNA methyltransferase. In preferred embodiments, only the newly synthesized stands (i.e., strands tagged by the tagged primer) are used and analyzed in the subsequent steps. FIG. 11 provides a schematic depiction of the use of tagged primers in the process. In this figure, “A” shows the conventional bisulfite conversion and sequencing assay and “B” shows the methyl transferase dependent assay. As shown in the left panel for assay “B”, the use of primer extension from a biotinylated primer and subsequent isolation with streptavidin beads ensures that all bottom strands in the analysis will be of the newly synthesized ones. Therefore, by performing DNMT1 (or other methyl transferase) treatment and next analyze the biotin-streptavidin isolated bottom strands one will get a direct and accurate quantification of the 5meC level of the complementary strand. Right panel, top, shows representative sequencing results of 10 clones for the standard bisulfite assay, “A”, where both 5meC and 5hmC will be read as cytosine after treatment, and representative sequencing results of 10 clones for the methyl transfer assay “B” where only 5meC will be read as a cytosine after treatment. The combination of the standard bisulfite assay data, “A”, where both 5meC and 5hmC will be read as cytosine after treatment and the methyl transfer assay “B” where only 5meC will be read as a cytosine after treatment allows for determination of position and quantity of 5hmC (from the simple calculation: A−B=5hmC). This quantification is outlined in the bottom of the right panel. For experimental replicates with this exact quantitative outcome we have n=15.

The present invention is not limited to the use of any particular tagged primer or tag binding reagent for isolation of the tagged primer. In some preferred embodiments, the primer is biotinylated and the tag binding reagent is a streptavidin reagent, such as a streptavidin bead. Replicated nucleic acid strands comprising the biotinylated primer (i.e., the primer extension product resulting from extension of the biotinylated primer) are isolated by contacting the strands with the streptavidin beads. Any combination of tagged primer and tag binding reagent may be utilized. Other suitable examples include haptenylated primers and beads or other reagents comprising an antibody or other antigen binding protein that binds to the hapten. Suitable haptens include, but are not limited to, pyrazoles, particularly nitropyrazoles; nitrophenyl compounds; benzofurazans; triterpenes; ureas and thioureas, particularly phenyl ureas, and even more particularly phenyl thioureas; rotenone and rotenone derivatives, also referred to herein as rotenoids; oxazole and thiazoles, particularly oxazole and thiazole sulfonamides; coumarin and coumarin derivatives; cyclolignans, exemplified by Podophyllotoxin and Podophyllotoxin derivatives; and combinations thereof. Specific examples of haptens include, but are not limited to, 2,4-Dintropheyl (DNP), Biotin, Fluorescein derivatives (FITC, TAMRA, Texas Red, etc.), Digoxygenin (DIG), 5-Nitro-3-pyrozolecarbamide (nitropyrazole, NP), 4,5,-Dimethoxy-2-nitrocinnamide (nitrocinnamide, NCA), 2-(3,4-Dimethoxyphenyl)-quinoline-4-carbamide (phenylquinolone, DPQ), 2,1,3-Benzoxadiazole-5-carbamide (benzofurazan, BF), 3-Hydroxy-2-quinoxalinecarbamide (hydroxyquinoxaline, HQ), 4-(Dimethylamino)azobenzene-4′-sulfonamide (DABSYL), Rotenone isoxazoline (Rot), (E)-2-(2-(2-oxo-2,3-dihydro-1H-benzo[b][1,4]diazepin-4-yl)phenozy)acetamide (benzodiazepine, BD), 7-(diethylamino)-2-oxo-2H-chromene-3-carboxylic acid (coumarin 343, CDO), 2-Acetamido-4-methyl-5-thiazolesulfonamide (thiazolesulfonamide, TS), and p-Mehtoxyphenylpyrazopodophyllamide (Podo).

In some embodiments, the 5hmC groups in the sample are modified with a blocking group to increase the ratio of methyl transferase efficiency between 5meC and 5hmC. As used herein, a “blocking group” is any chemical group that can be added to 5hmC (or cytosine at the 5-carbon position) that makes the total group too large, or unfavorably charged, for the DNA methyl transferase pocket, and thus blocks activity of a DNA methyl transferase at the 5hmC residue. It is contemplated that use of blocking groups increases the ratio of DNMT1 methyl transferase specificity and/or efficiency for catalyzing the transfer of a methyl group across from a 5meC and 5hmC in dsDNA. The present invention is not limited to the use of any particular blocking group. Suitable blocking groups include, but are not limited to Glucose (beta-glucose and alpha-glucose); Gentiobiose (6-O-β-D-glucopyranosyl-D-glucose) (and any other stereoisomer, the alpha linkage is also possible: 6-O-alpha-D-glucopyranosyl-D-glucose); keto-glucose; azide-glucose (e.g. N3Glucose); a chemical group linked to the glucose or azide-glucose by, e.g., click chemistry, for example biotin (biotin-N3Glucose-5hmC); JBP1 (J-binding protein 1) bound to glu-5hmC (full length and truncated versions); TET proteins (e.g. TET1, TET2 and TET3) (full length and truncated versions) bind to 5hmC; other 5hmC or Glu-5hmC binding proteins and/or protein binding domains; (native and cross-linked versions of proteins); any oxidation product of glucose or modified glucose e.g. periodate oxidized glucose; any chemical group that can react with oxidized glucose to bind to or modify the glucose; and any protein or protein complex that can specifically identify either 5meC, 5hmC and modified variants of these bases (e.g., JBP1 and proteins of the MBP class (e.g., MBP1 and MeCP2)).

Without blocking, it is possibly to achieve 100% vs 0% vs 0% methyl transfer across from 5meC, C and 5hmC respectively, although the method is also applicable at less than 100% methyl transfer across from 5meC and more than 0% transfer across from C and 5hmC. Increased accuracy in quantification in such cases can be obtained when a known control is spiked into the sample so that the in-sample efficiency can be determined. With blocking it is possible to achieve 100% vs 0% vs 0% methyl transfer across from 5meC, C and 5hmC respectively, although the method is also applicable at less than 100% methyl transfer across from 5meC and more than 0% transfer across from C and 5hmC. Blocking may be useful for the “standard” assay as this will allow one to more robustly achieve 100% vs 0% vs 0% with the DNMT1 assay at a higher success rate as compared to without blocking.

With blocking, a 100% vs 100% vs 0% methyl transfer across from 5meC, C, 5hmC respectively for M.SssI (or DNMT1, preferably a large molar excess of DNMT1) is achievable. Methylation across from 5meC and C is an alternative way to transfer the information of the modification status from the parent strand to the replicated/primer extended strand to help in identifying 5meC, 5hmC and C positions and quantities. This can enable the direct read out of 5hmC as unmodified cytosines which are not protected from bisulfite conversion, or in comparison to standard bisulfite conversion and sequencing can reveal quantitative information for 5meC, C and 5hmC in the nucleic acid sequence. This will allow for the simple calculation to reveal the position and quantity of each of 5meC, C and 5hmC.

It is likely that 5meC, 5hmC and C identification can be achieved if blocking is performed at both 5hmC and C residues. Blocking agents at cytosine residues could for example be CXXC motif containing proteins or any protein or fragment thereof which can bind to unmodified CpG.

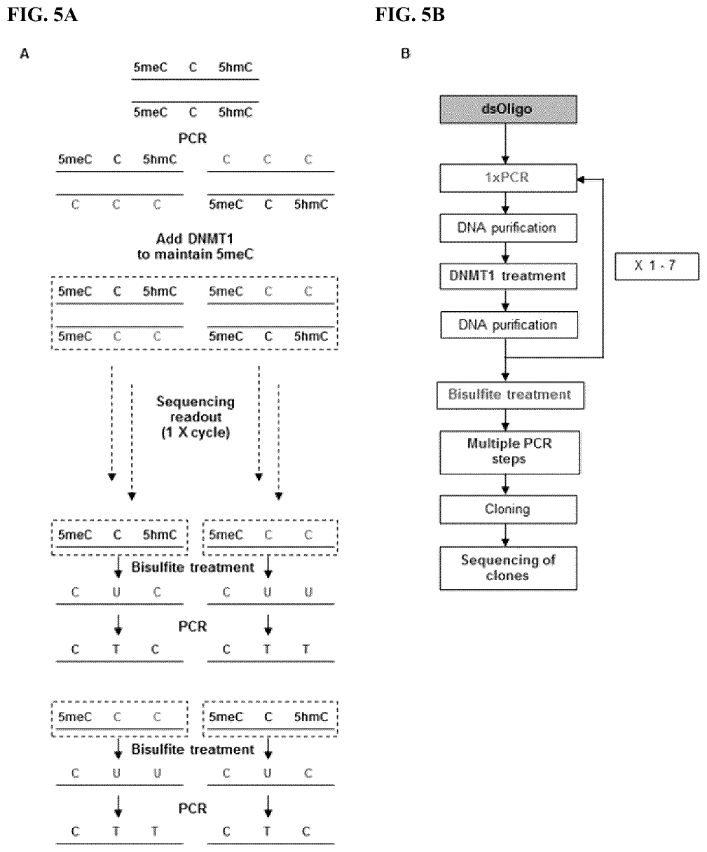

In some embodiments, the 5hmC diluted nucleic acid sample and an undiluted portion are treated to convert unmodified cytosine residues to thymidine residues. In preferred embodiments, the portions are treated with bisulfite to convert unmodified cytosine residues to uracil residues. The bisulfite-treated nucleic acid is then replicated with a polymerase to convert said uracil residues into thymidine residues. In some embodiments, the replication step is performed via one or more rounds of polymerase chain reaction (see, e.g., FIGS. 1 and 5) or primer extension reaction (See, e.g., FIG. 5). In preferred embodiments, a predetermined region is replicated by extension from nucleic acid primers defining the 5′ and 3′ boundaries of the predetermined region. In some embodiments, the number of cycles of replication may be greater than 2, 3, 5, 7, 10 or 20 cycles or between about 2 and 20 cycles.

The process described in the preceding paragraphs provides two different nucleic acid portions. In the first portion, the 5-hydroxymethylated residues have been diluted as compared to the 5-methylated cytosine residues, which have been maintained. In the second portion, the 5-hydroxymethylated residues have not been diluted. When the portions are treated with bisulfite, all non-modified cytosine residues are converted to uracil residues and then to thymidine residues following the 1 or more rounds of replication or primer extension. In preferred embodiments, both portions are sequenced, preferably utilizing primers that allow sequencing of the predetermined region. In preferred embodiments, comparison of the sequences of the first and second portions allow identification of 5meC and 5hmC residues in the predetermined region. 5hmC residues are identified as residues that are read by sequencing as a thymidine residue in the first portion (i.e., the portion in which 5hmC residues have been diluted) and as a cytosine residue at the corresponding position in the second nucleic acid portion and 5meC residues are identified as residues that are read as cytosine residues in both of the first and second nucleic acid portions.

Sequencing of the nucleic acid samples may be performed by any method known in the art. Suitable sequencing methods include, but are not limited to, chain termination sequencing methods (e.g., Sanger sequencing methods) and nextgen DNA sequencing methods utilizing systems provides by Illumina (San Diego CA), Pacific Biosciences (Menlo Park, CA) and others. In embodiments using nextgen sequencing methods, the step of replicating with a polymerase prior to sequencing (which converts the uracil residue to a thymidine residue) is optional and the uracil residue may be read directly.

In some embodiments, the processes described above are utilized for predicting a predisposition to a disease in a subject, diagnosing a disease in a subject, predicting the likelihood of recurrence of disease in a subject, providing a prognosis for a subject with a disease, or selecting a subject with a disease for treatment with a particular therapy. These process preferably comprise providing a genomic DNA sample from a subject; and detecting the methylation status of predetermined regions of the genomic DNA sample by the processes described above. In some embodiments, an altered level of 5-hydroxymethylcytosine and/or 5-methylcytosine methylation (i.e., a higher or lower level) of the predetermined regions of the genomic DNA to a reference methylation status provides an indication selected from the group consisting of an indication of a predisposition of the subject to a disease, an indication that the subject has a disease, an indication of the likelihood of recurrence of a disease in the subject, an indication of survival of the subject, and an indication that the subject is a candidate for treatment with a particular therapy.

Accordingly, in some embodiments, methods of the present invention involve the determination (e.g., assessment, ascertaining, quantitation) of 5meC and/or 5hmC modification level of an indicator of a condition of interest, such as a neoplasm in a sample. A skilled artisan understands that an increased, decreased, informative, or otherwise distinguishably different 5meC and/or5hmC modification level is articulated with respect to a reference (e.g., a reference level, a control level, a threshold level, or the like). For example, the term “elevated 5hmC or 5meC level” as used herein with respect to the 5hmC or 5meC status of a gene locus is any 5hmC and/or 5meC level that is above a median 5hmC or 5meC level in a sample from a random population of mammals (e.g., a random population of 10, 20, 30, 40, 50, 100, or 500 mammals) that do not have a neoplasm (e.g., a cancer) or other condition of interest. Elevated levels of 5meC and/or 5hmC modification can be any level provided that the level is greater than a corresponding reference level. For example, an elevated 5meC and/or 5hmC level of a locus of interest can be 0.5, 1, 2, 3, 4, 5, 6, 7, 8, 9, 10, or more fold greater than the reference level 5meC and/or 5hmC observed in a normal sample. It is noted that a reference level can be any amount. The term “elevated 5meC and/or 5hmC score” as used herein with respect to detected 5meC and/or 5hmC events in a matrix panel of particular nucleic acid markers is any 5meC and/or 5hmC score that is above a median 5meC and/or 5hmC score in a sample from a random population of mammals (e.g., a random population of 10, 20, 30, 40, 50, 100, or 500 mammals) that do not have a neoplasm (e.g., a cancer). An elevated 5hmC score in a matrix panel of particular nucleic acid markers can be any score provided that the score is greater than a corresponding reference score. For example, an elevated score of 5meC and/or 5hmC in a locus of interest can be 0.5, 1, 2, 3, 4, 5, 6, 7, 8, 9, 10, or more fold greater than the reference 5meC and/or 5hmC score observed in a normal sample. It is noted that a reference score can be any amount that is used for comparison.

Similar considerations apply to assays for decreased levels of 5meC and/or 5hmC modifications in a sample, target locus, target genomic region and the like. For example, the term “decreased 5meC and/or 5hmC level” as used herein with respect to the 5meC and/or 5hmC status of a gene locus is any 5meC and/or 5hmC level that is below a median 5meC and/or 5hmC level in a sample from a random population of mammals (e.g., a random population of 10, 20, 30, 40, 50, 100, or 500 mammals) that do not have a neoplasm (e.g., a cancer). Decreased levels of 5meC and/or 5hmC modification can be any level provided that the level is less than a corresponding reference level. For example, a decreased 5meC and/or 5hmC level of a locus of interest can be 0.5, 1, 2, 3, 4, 5, 6, 7, 8, 9, 10, or more fold less than the reference level 5meC and/or 5hmC observed in a normal sample. It is noted that a reference level can be any amount. The term “decreased 5hmC score” as used herein with respect to detected 5meC and/or 5hmC events in a matrix panel of particular nucleic acid markers is any 5meC and/or 5hmC score that is below a median 5meC and/or 5hmC score in a sample from a random population of mammals (e.g., a random population of 10, 20, 30, 40, 50, 100, or 500 mammals) that do not have a neoplasm (e.g., a cancer). A decreased 5meC and/or 5hmC score in a matrix panel of particular nucleic acid markers can be any score provided that the score is greater than a corresponding reference score. For example, a decreased score of 5meC and/or 5hmC in a locus of interest can be 0.5, 1, 2, 3, 4, 5, 6, 7, 8, 9, 10, or more fold less than the reference 5meC and/or 5hmC score observed in a normal sample. It is noted that a reference score can be any amount that is used for comparison.

The methods are not limited to a particular type of mammal. In some embodiments, the mammal is a human. In some embodiments, the neoplasm is premalignant. In some embodiments, the neoplasm is malignant. In some embodiments, the neoplasm is cancer without regard to stage (e.g., stage I, II, III, or IV).

The present invention also provides methods and materials to assist medical or research professionals in determining whether or not a mammal has a neoplasm (e.g., cancer). Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the ratio of 5hmC and/or other markers in a sample, and (2) communicating information about the ratio to that professional, for example.

After the level (e.g., score or frequency) of particular 5meC and/or 5hmC modification in a sample is reported, a medical professional can take one or more actions that can affect patient care. For example, a medical professional can record the results in a patient's medical record. In some cases, a medical professional can record a diagnosis of a neoplasia, or otherwise transform the patient's medical record, to reflect the patient's medical condition. In some cases, a medical professional can review and evaluate a patient's entire medical record, and assess multiple treatment strategies, for clinical intervention of a patient's condition. In some cases, a medical professional can record a prediction of tumor occurrence with the reported indicators. In some cases, a medical professional can review and evaluate a patient's entire medical record and assess multiple treatment strategies, for clinical intervention of a patient's condition.

A medical professional can initiate or modify treatment of a neoplasm after receiving information regarding the level (score, frequency) associated with 5meC and/or 5hmC level in a patient's urine sample. In some cases, a medical professional can compare previous reports and the recently communicated level (score, frequency) of 5meC and/or 5hmC modification, and recommend a change in therapy. In some cases, a medical professional can enroll a patient in a clinical trial for novel therapeutic intervention of neoplasm. In some cases, a medical professional can elect waiting to begin therapy until the patient's symptoms require clinical intervention.

A medical professional can communicate the assay results to a patient or a patient's family. In some cases, a medical professional can provide a patient and/or a patient's family with information regarding neoplasia, including treatment options, prognosis, and referrals to specialists, e.g., oncologists and/or radiologists. In some cases, a medical professional can provide a copy of a patient's medical records to communicate assay results to a specialist. A research professional can apply information regarding a subject's assay results to advance neoplasm research. For example, a researcher can compile data on the assay results, with information regarding the efficacy of a drug for treatment of neoplasia to identify an effective treatment. In some cases, a research professional can obtain assay results to evaluate a subject's enrollment, or continued participation in a research study or clinical trial. In some cases, a research professional can classify the severity of a subject's condition, based on assay results. In some cases, a research professional can communicate a subject's assay results to a medical professional. In some cases, a research professional can refer a subject to a medical professional for clinical assessment of neoplasia, and treatment thereof. Any appropriate method can be used to communicate information to another person (e.g., a professional). For example, information can be given directly or indirectly to a professional. For example, a laboratory technician can input the assay results into a computer-based record. In some cases, information is communicated by making a physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

It is noted that a single sample can be analyzed for one neoplasm-specific marker or for multiple neoplasm-specific markers. In preferred embodiments, a single sample is analyzed for multiple neoplasm-specific markers, for example, using multi-marker assays. In addition, multiple samples can be collected for a single mammal and analyzed as described herein. In some embodiments, a sample is split into first and second portions, where the first portion undergoes cytological analysis and the second portion undergoes further purification or processing (e.g., sequence-specific capture step(s) (e.g., for isolation of specific loci for analysis of 5hmC levels). In some embodiments, the sample undergoes one or more preprocessing steps before being split into portions. In some embodiments, the sample is treated, handled, or preserved in a manner that promotes DNA integrity and/or inhibits DNA degradation (e.g., through use of storage buffers with stabilizing agents (e.g., chelating agents, DNase inhibitors) or handling or processing techniques that promote DNA integrity (e.g., immediate processing or storage at low temperature (e.g., −80 degrees C.)).

In some embodiments, all the basic essential materials and reagents required for detecting neoplasia through detecting both the level (presence, absence, score, frequency) of markers in a sample obtained from the mammal are assembled together in a kit. Such kits generally comprise, for example, reagents useful, sufficient, or necessary for detecting and/or characterizing one or more markers (e.g., epigenetic markers; 5hmC modifications) specific for a neoplasm. In some embodiments, the kits contain enzymes suitable for amplifying nucleic acids including various polymerases, deoxynucleotides and buffers to provide the necessary reaction mixture for amplification. In some embodiments, the kits of the present invention include a means for containing the reagents in close confinement for commercial sale such as, e.g., injection or blow-molded plastic containers into which the desired reagent are retained. Other containers suitable for conducting certain steps of the disclosed methods also may be provided.

In some embodiments, the methods disclosed herein are useful in monitoring the treatment of neoplasia (e.g., cancer). For example, in some embodiments, the methods may be performed immediately before, during and/or after a treatment to monitor treatment success. In some embodiments, the methods are performed at intervals on disease free patients to ensure treatment success.

The present invention also provides a variety of computer-related embodiments. Specifically, in some embodiments the invention provides computer programming for analyzing and comparing a pattern of neoplasm-specific marker detection results in a sample obtained from a subject to, for example, a library of such marker patterns known to be indicative of the presence or absence of a neoplasm, or a particular stage or neoplasm.

In some embodiments, the present invention provides computer programming for analyzing and comparing a first and a second pattern of neoplasm-specific marker detection results from a sample taken at least two different time points. In some embodiments, the first pattern may be indicative of a pre-cancerous condition and/or low risk condition for cancer and/or progression from a pre-cancerous condition to a cancerous condition. In such embodiments, the comparing provides for monitoring of the progression of the condition from the first time point to the second time point.

In yet another embodiment, the invention provides computer programming for analyzing and comparing a pattern of neoplasm-specific marker detection results from a sample to a library of neoplasm-specific marker patterns known to be indicative of the presence or absence of a cancer, wherein the comparing provides, for example, a differential diagnosis between a benign neoplasm, and an aggressively malignant neoplasm (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of neoplasm-specific marker (e.g., epigenetic marker, 5hmC modification) detection results from a sample). This information received can be stored at least temporarily in a database, and data analyzed in comparison to a library of marker patterns known to be indicative of the presence or absence of a pre-cancerous condition, or known to be indicative of a stage and/or grade of cancer.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of neoplasm-specific marker detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a sample obtained from a subject is to be input by a user (e.g., a technician or someone performing the assays)) and transmitted to a remote site to a second computer processor for analysis (e.g., where the pattern of neoplasm-specific marker) detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of a neoplasm, or monitoring the progression of a pre-cancerous condition or a neoplasm. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of a neoplasm, or a relational database (RDB) which can include data input by the user and data output. The computer processors can be processors that are typically found in personal desktop computers (e.g., IBM, Dell, Macintosh), portable computers, mainframes, minicomputers, or other computing devices.

The input components can be complete, stand-alone personal computers offering a full range of power and features to run applications. The user component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe, or distributed across multiple servers (e.g., as in cloud computing applications) and offers data management, information sharing between clients, network administration and security. The application and any databases used can be on the same or different servers. Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

The present invention is useful for both the diagnosing diseases and disorders in a subject as well as determining the prognosis of a subject. The methods, reagents and systems of the present invention are applicable to a broad variety of diseases and disorders. In certain embodiments, the present invention provides methods for obtaining a subject's risk profile for developing neoplasm (e.g., cancer). In some embodiments, such methods involve obtaining a sample from a subject (e.g., a human at risk for developing cancer; a human undergoing a routine physical examination), detecting the presence, absence, or level (e.g., 5hmC modification frequency or score) of one or more markers specific for a neoplasm in or associated with the sample (e.g., specific for a neoplasm) in the sample, and generating a risk profile for developing neoplasm (e.g., cancer) based upon the detected level (score, frequency) or presence or absence of the indicators of neoplasia. For example, in some embodiments, a generated risk profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific 5hmC modifications as determined with the methods of the present invention. In some embodiments, the presence and absence of specific markers as determined with the methods of the present invention are inputted into such an algorithm, and the risk profile is reported based upon a comparison of such input with established norms (e.g., established norm for pre-cancerous condition, established norm for various risk levels for developing cancer, established norm for subjects diagnosed with various stages of cancer). In some embodiments, the risk profile indicates a subject's risk for developing cancer or a subject's risk for re-developing cancer. In some embodiments, the risk profile indicates a subject to be, for example, a very low, a low, a moderate, a high, and a very high chance of developing or re-developing cancer. In some embodiments, a health care provider (e.g., an oncologist) will use such a risk profile in determining a course of treatment or intervention (e.g., biopsy, wait and see, referral to an oncologist, referral to a surgeon, etc.).

Other diseases and disorders that may be diagnosed or prognosed with the methods, reagents and systems of the present invention include, but are not limited to, Prader-Willi syndrome, Angelman syndrome, Beckwith-Wiedemann syndrome, Pseudohypoparathyroidism, Russell-Silver syndrome, ICF syndrome, Rett syndrome, α-thalassemia/mental retardation, X-linked (ATR-X), Immunoosseous dysplasia, Schimke type, Rubinstein-Taybi syndrome, MTHFR deficiency, Recurrent hydatidiform mole, Fragile X mental retardation syndrome,

Deletion LCR γδβ- and δβ-thalassemia, FSH dystrophy, disorders of XIC, Schimke immunoosseous dysplasia (SIOD), Sotos syndrome, Atrichia, X-linked Emery-Dreifuss muscular dystrophy (EDMD), Autosomal EDMD, CMT2B1, mandibuloacral dysplasia, limb-girdle muscular dystrophy type 1B, familial partial lipodystrophy, dilated cardiomyopathy 1A, Hutchinson-Gilford progeria syndrome, and Pelger-Huet anomaly.

The following examples are provided in order to demonstrate and further illustrate certain preferred embodiments and aspects of the present invention and are not to be construed as limiting the scope thereof.

### EXAMPLE 1

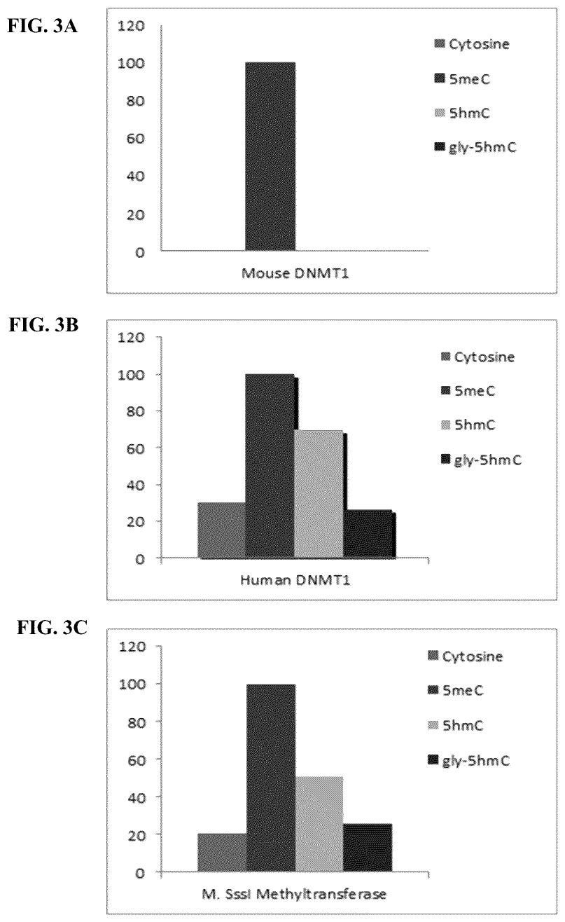

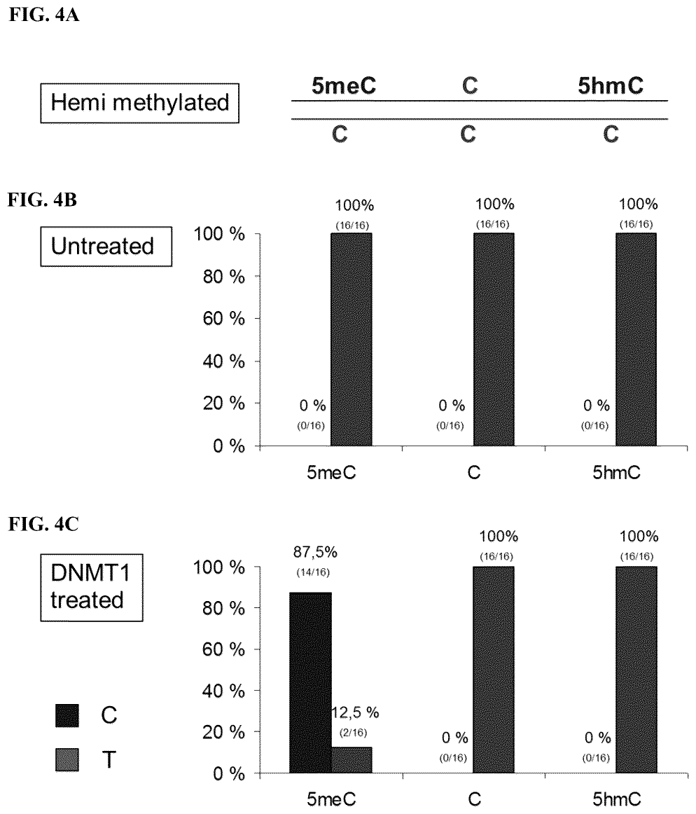

The method described here relies on the successive dilution by PCR of the 5hmC modification along with maintenance of the 5meC modification given that DNMT1 cannot methylate across from 5hmC and cytosine; however, DNMT1 can methylated DNA across from 5meC. FIGS. 3 and 4 demonstrate that DNMT1 cannot catalyze the transfer of a methyl group from S-adenosyl-methylmethionine when the DNA substrate is either a cytosine, 5hmC or a β-glucosyl-5hmC. Therefore, it is possible to dilute the 5hmC modification by PCR followed by treatment with DNMT1 while the 5meC modification will be maintained through multiple rounds of PCR and DNMT1 treatment.

Our method applies bisulfite conversion and sequencing of sample “A”, untreated DNA, which will be used as a reference as it will detect the total of both 5meC and 5hmC. The method involves a 5hmC dilution assay, diluting 5hmC in the total pool of DNA fragments while maintaining 5meC. This dilution is achieved through sequential rounds of one cycle of PCR amplification (dilution) and treatment of the DNA with the DNA maintenance methyltransferase DNMT1 which enzymatically and specifically maintains 5meC by adding a methyl group uniquely to the unmethylated strand of the hemimethylated PCR products (this sample is referred to as sample “B” in FIG. 1). After a few rounds of this assay we apply bisulfite conversion and sequencing of the treated DNA sample, sample B, where 5meC now is the only modification present (or the only modification highly maintained). Therefore, all (or most) bases that read as C from this sample must have been protected against conversion because of 5meC and not 5hmC. By comparing “B” to the reference sample “A” we can easily detect all base positions containing 5hmC.

It should be noted that this method while effectively diluting 5hmC, it maintains the 5meC signal. Therefore, this method can serve two purposes (i) the identification of 5hmC in DNA and (ii) the identification of 5meC in DNA. The proof of feasibility of the assay described above is demonstrated in FIG. 4.

### Experimental Design

The method described here relies on the successive dilution by PCR of the 5hmC modification along with maintenance of the 5meC modification given that DNMT1 cannot methylate across from 5hmC and cytosine; however, DNMT1 can methylate DNA across from 5meC. FIGS. 3. A and 4 demonstrate that DNMT1 cannot catalyze the transfer of a methyl group from S-adenosyl-methylmethionine when the DNA substrate is either a cytosine, 5hmC or a b-glucosyl-5hmC. Therefore, it is possible to dilute the 5hmC modification by PCR followed by treatment with DNMT1 while the 5meC modification will be maintained through multiple rounds of PCR and DNMT1 treatment. Importantly, DNMT1 enzymes as well as other methyltransferases could be employed to distinguish between 5meC and 5hmC even when these enzymes do methylate across from 5hmC and cytosine as long the enzymes have a preference for the hemi 5meC over 5hmC, b-glucosyl-5hmC or cytosine at CpG sites (FIG. 3. B and C). Moreover, a DNMT1 or methyltransferase enzyme can allow for the identification of both 5meC and 5hmC in the assay described here even if the transfer of a methyl group from S-adenosyl-methylmethionine is of a much lower rate than 100%. The requirement for distinguishing 5meC from 5hmC is that there is a preference for 5meC over 5hmC, b-glucosyl-5hmC or cytosine at CpG sites (FIG. 3B and C).

Our method applies bisulfite conversion and sequencing of sample “A”, untreated DNA (FIG. 1), which will be used as a reference as it will detect the total of both 5meC and 5hmC. The method involves a 5hmC dilution assay, diluting 5hmC in the total pool of DNA fragments while maintaining 5meC. This dilution is achieved through sequential rounds of one cycle of PCR amplification (dilution) and treatment of the DNA with the DNA maintenance methyltransferase DNMT1 which enzymatically and specifically maintains 5meC by adding a methyl group uniquely to the unmethylated strand of the hemimethylated PCR products (this sample is referred to as sample “B” in FIG. 1). After a few rounds of this assay we apply bisulfite conversion and sequencing of the treated DNA sample, sample B, where 5meC now is the only modification present (or the only modification highly maintained). Therefore, all (or most) bases that read as C from this sample must have been protected against conversion because of 5meC and not 5hmC. By comparing “B” to the reference sample “A” we can easily detect all base positions containing 5hmC.

It should be noted that this method while effectively diluting 5hmC, it maintains the 5meC signal. Therefore, this method can serve two purposes (i) the identification of 5hmC in DNA and (ii) the identification of 5meC in DNA. The proof of feasibility of the assay described above is demonstrated in FIG. 4.

Furthermore, it should be noted that the current state of the art bisulfite conversion kits has limitations in the sensitivity. For example, the MethylEasy Xceed kit (Human Genetic Signatures, cat. no. ME002) allows for the analysis of 5meC from as few as 8 cells, but does not allow for single cell analysis. The method described here will while effectively diluting 5hmC and maintaining the 5meC signal allow for increased sensitivity of detection of both 5meC and 5hmC, with an obvious potential for single cell analysis as a result of the PCR amplification of the DNA sample (with either gene specific or whole genome amplification).

### MATERIALS AND METHODS

### Substrates

DNA substrates created by annealing the appropriate complementary oligonucleotide (see Supplemental Table 1) by heating to 95° C. and cooling at 1° C./min until the reaction reached 25° C. The methyltransferase specificity assay utilized oligonucleotides created by annealing either 5hmC top, 5meC top or cytosine top with cytosine bottom. The substrate used to simulate one round of PCR followed by DNMT1 treatment was created by annealing 5hmC:C:5meC top with unmodified bottom. The substrate used for the full assay was created by annealing 5hmC:C:5meC top with 5hmC:C:5meC bottom.

### Methyltransferase Specificity Assay

Reactions (50 μl) containing 100 ng DNA substrate (either cytosine, hemi-5meC, or hemi-5hmC), 50 mM Tris-HCl, 1 mM Dithiothreitol, 1 mM EDTA pH 8.0, 5% (v/v) Glycerol, S-[methyl-¹⁴C]-Adenosyl-L-Methionine were incubated at 37° C. with 2 units of recombinant mouse DNMT1, recombinant human DNMT1, or SssI Methyltransferase for 30 minutes. Reactions were terminated by the addition of 200 μl TE buffer. The DNA from the reactions was ethanol precipitated and washed three times with ice-cold 70% ethanol. The DNA pellets were dried and suspended in 20 μl TE buffer. The entire reaction was transferred into a 5 ml Scintillation vial containing 2 ml Ecosinct A. The acid insoluble fractions were scintillation counted using an open window for 10 minutes.

### Bisulfite Conversion, Cloning and Sequencing

Bisulfite conversion was carried out according to the user guide of the MethylEasy Xceed kit (Human Genetic Signatures, cat. no. ME002). Cloning was performed using the TOPO TA kit (Invitrogen, cat. no. K4595-40). Sequencing was carried out using the method described by Sanger.

### Proof of Principle for the 5hmC Dilution Assay

A 112 bp dsDNA oligo containing three CpG sites where one is hemi-5meC, the second CpG contained no modification and the third CpG was hemi-5hmC, was used for a proof of principle of the 5hmC dilution assay. The oligo (65 ng) was added to a mixture of 5.0 μl 10× DNMT1-buffer (NEB), 2.5 μl of 3.2 mM SAM, 0.5 μl BSA (NEB, cat. no. B9001S) and 10 Units of mouse DNMT1 in a total volume of 50 μl adjusted with MqH₂O. The DNMT1 reactions were incubated on a Thermomixer at 37° C., 600 rpm for 4 h. The DNA oligoes were subsequently purified with a MinElute Reaction Cleanup Kit. Bisulfite conversion and sequencing of the unmodified bottom strand of the oligo was carried out before and after DNMT1 treatment.

### The 5hmC Dilution Assay

A 112 bp dsDNA oligo containing three CpG sites, one having 5meC at both strands, a second one having no modification and a third one having 5hmC at both strands, was used to demonstrate the 5hmC dilution assay. To make hemi-modified oligonucleotides, PCR was set up and ran as following: The oligonucleotide (65 ng) was added to a mixture of 4.0 μl of 5× Phusion HF-buffer, 1.6 μl of 2.5 mM dNTPs, 1 μl of each of 10 μM forward and reverse primers, 0.2 μl of Phusion polymerase in a total volume of 20 μl adjusted with MqH₂O. Melting of the DNA strands was carried out for 3 min at 98° C., followed by primer annealing for 2 min at 56° C. and elongation for 8 min at 72° C. Next, the DNA was purified with a MinElute Reaction Cleanup Kit, the concentration was measured fluorimetrically on a Qubit instrument and DNMT1 treatment was carried out according to the following set up: The total amount of recovered oligo was added to a mixture of 5.0 μl 10× DNMT1-buffer (NEB), 2.5 μl of 3.2 mM SAM, 0.5 μl BSA (NEB, cat. no. B9001S) and 10 Units of mouse DNMT1 in a total volume of 50 μl adjusted with MqH₂O. The DNMT1 reactions were incubated on a Thermomixer at 37° C., 600 rpm for 4 h. Subsequently, 1 μl of Proteinase K was added (14-22 mg/ml) (Roche) and further incubation was carried out at 50° C. on a Thermomixer, 600 rpm for 1 h. The DNA oligoes was then ethanol precipitated and further purified with a MinElute Reaction Cleanup Kit. The DNA concentration was again measured fluorimetrically on a Qubit instrument. The setup described in this section can be carried out one or more times to result in a range of 5hmC dilution and 5meC conservation.

### The 5hmC Dilution/Loss Assay Allowing for Strand Specific Assessment

A 112 bp dsDNA oligo containing three CpG sites, one having 5meC at both strands, a second one having no modification and a third one having 5hmC at both strands, was used to demonstrate the 5hmC dilution assay (also referred to as 5hmC loss assay and (biotin-) primer extension assay) making use of strand specific assessment. To make hemi-modified oligonucleotides, strand specific primer extension PCR was set up and ran as following: The oligonucleotide (65 ng) was added to a mixture of 4.0 μl of 5× Phusion HF-buffer, 1.6 μl of 2.5 mM dNTPs, 1 μl of only one of 10 μM forward and reverse primers containing a 5′ biotin molecule/tag, 0.2 μl of Phusion polymerase in a total volume of 20 μl adjusted with MqH₂O. Melting of the DNA strands was carried out for 3 min at 98° C., followed by primer annealing for 2 min at 56° C. and elongation for 8 min at 72° C. Next, the DNA was purified with a Streptavidine coated magnetic beads and DNMT1 treatment was carried out according to the following set up: The total amount of recovered oligo was added to a mixture of 5.0 μl 10× DNMT1-buffer (NEB), 2.5 μl of 3.2 mM SAM, 0.5 μl BSA (NEB, cat. no. B9001S) and 10 Units of mouse DNMT1 in a total volume of 50 μl adjusted with MqH₂O. The DNMT1 reactions were incubated on a Thermomixer at 37° C., 600 rpm for 4 h. The biotinylated oligonucleotides were subsequently purified by using streptavidine magnetic beads and bisulfate converted, used as templates in PCR and sequenced.

### RESULTS

An outline the method is demonstrated in FIG. 1. To demonstrate the feasibility and success of the method we will demonstrate that (i) specific methyltransferases preferentially modify hemi-5meC DNA substrates, (ii) that this preference can be identified by bisulfite sequencing after treatment with the appropriate methyltransferase and (iii) the 5hmC modification can be diluted by successive rounds of DNA amplification followed by treatment with the appropriate DNA methylase.

### Mouse DNMT1, Human DNMT1, and the M. SssI Methyltransferase Preferentially Methylate Hemi-5meC Substrates

DNMT1 from mouse, DNMT1 from human and M. SssI methyltransferase were incubated with 100 ng of either unmodified, hemi-5meC, hemi-5hmC or hemi-beta-glucosyl-5hmC. Mouse DNMT1 was able to catalyze the transfer of a methyl group exclusively to the hemi-5meC substrate, showing no activity on the other substrates (FIG. 3A). Human DNMT1 shows an enzymatic preference for hemi-5meC while showing limited activity on the other substrates (FIG. 3B). Finally, the M. SssI methylase (Spiroplasma sp.) also showed a preference for hemi-5meC containing DNA; (FIG. 3C). This result led us to the conclusion that any of these methyltransferases could suffice for the dilution assay described in FIG. 1.

### Mouse DNMT1 Strongly Prefers Hemi-5meC as a Substrate

A dsDNA substrate containing a hemi-5meC, unmodified cytosine, and hemi-5hmC was incubated with mouse DNMT1 in the presence of S-adenosyl methylmethionine. DNA was cleaned and subjected to bisulfite sequencing as described in “Materials and Methods.” After bisulfite sequencing we were able to demonstrate that nearly all (87.5%) of the hemi-5meC were fully methylated while the unmodified CpG and the hemi-5hmC were not modified by mouse DNMT1 (FIG. 4). As the substrate used for this assay mimics the fully 5hmC or fully-5hmC DNA after one round of amplification we determined that this assay would work if used with multiple rounds of DNA amplification and mouse DNMT1 treatment.

### Successive Rounds of DNMT1 Treatment and PCR Amplification can Dilute 5hmC while 5meC is Maintained

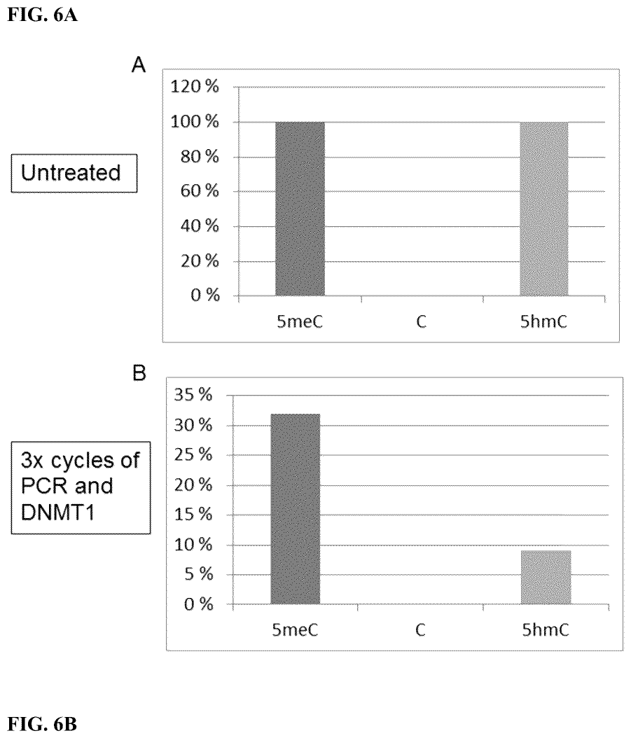

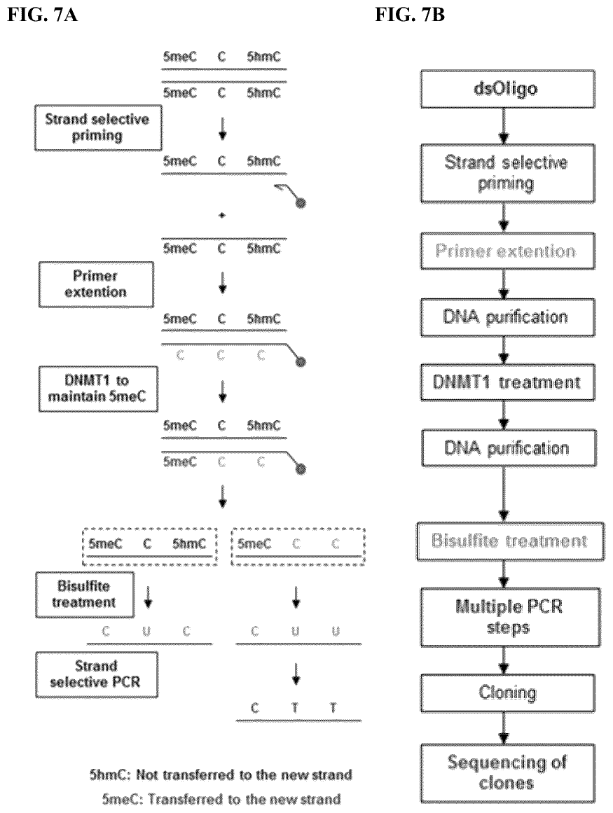

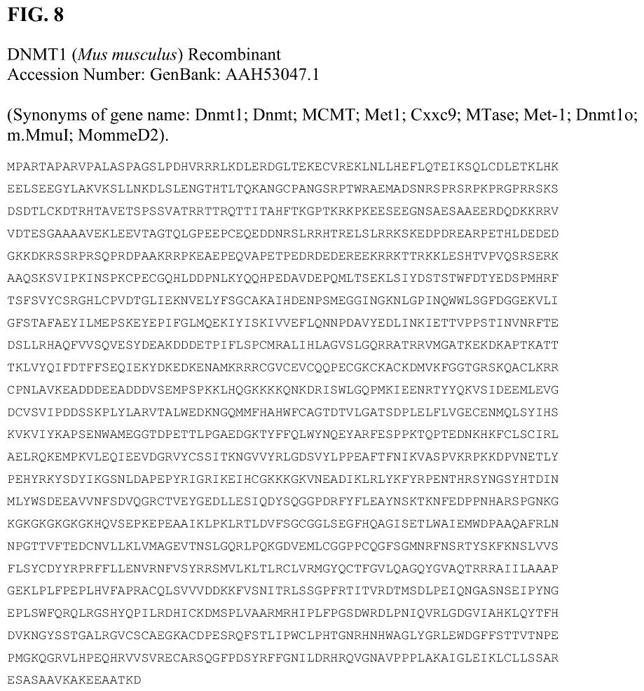

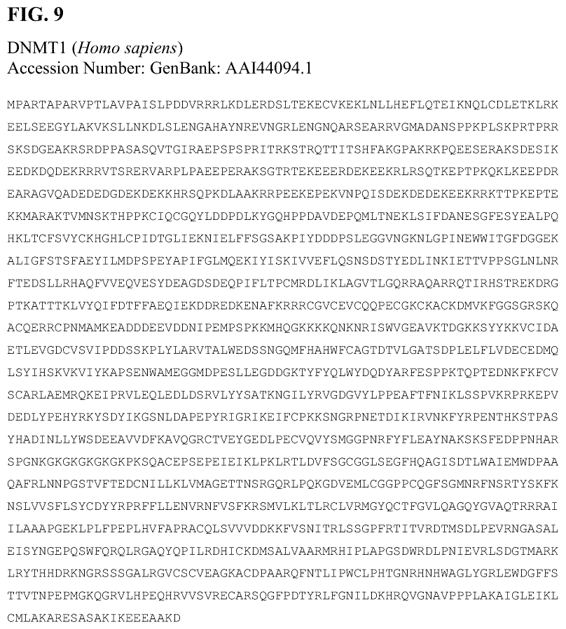

A dsDNA substrate containing a fully-5meC, CpG, and fully-5hmC was amplified using Taq or Phusion polymerase followed by treatment with mouse DNMT1. This procedure was carried out three times as described in “materials and methods.” FIG. 6B demonstrates the effective dilution of 5hmC while maintaining 5meC. It can be seen that prior to the dilution (FIG. 6A) the identity of 5hmC and 5meC cannot be distinguished; however, after the dilution treatment (FIG. 6B); 5hmC and 5meC can be clearly distinguished as the 5hmC is present at a greatly reduced amount compared to 5meC.

### Strand Specific Primer Extension PCR Combined with the Use of Biotinylated Primers Allows for the Assessment of the Rate of DNMT1 Transfer of Methyl Groups to CpG Sites on a Newly Synthesized Strand at Sites Across from 5hmC, 5meC or C

A dsDNA substrate containing a fully-5meC, CpG, and fully-5hmC was used as a template for strand specific primer extension PCR with primers containing a 5′ biotin tag and followed by treatment with mouse DNMT1. The oligonucleotides that was newly synthesized was isolated to make sure that any methyation/signal at the three CpG sites of analysis of the strand chosen for study would not come from the parental copy of the same strand. The strategy allows for direct detection and quantification of the 5meC and 5hmC level without further rounds of the assay. Such an assay containing the oligonucleotide described in the materials and methods section can also be used as an internal reference and control to aid in the calculation of the content of modified C bases in genomic DNA samples.

A representative protocol for the methyl transferase dependent assay (assay “B” in FIG. 11) is as follows:
Biotin-primer extension: “One round” PCR w/biotinylated primer
Pool PCR-products (from control oligo and genomic sample)
MinElute PCR Purification
Biotin-streptavidin purification with MyOne™ Streptavidin T1 beads
DNMT1 treatment on beads, 0.6-1 μl of 0.5 mg/ml DNMT1, 1.6 mM SAM, 37° C. in 30 min.
Washes; and elution in 50 μl MQ-H₂O 95° C. in 10 min.
MinElute Reaction Cleanup (optional)
Bisulfite treatment
Bisulfite PCR
TOPO TA cloning, transformation, selection on LB amp X-gal plates
Sequencing

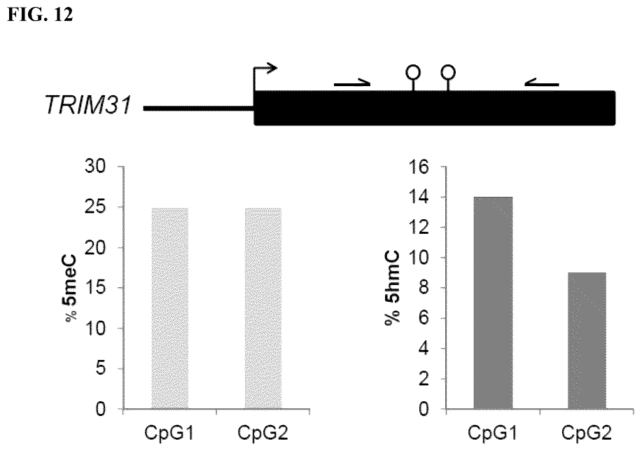

As shown in FIG. 12, the methyltransferase/DNMT1 dependent HyLo assay identified two 5hmC CpGs in the TRIM31 gene in human brain DNA. The assay outlined in FIG. 11 was used with genomic DNA spiked in with a control oligo containing known CpG sites for each of 5meC, C and 5hmC. Thus we could ensure accurate quantification of genomic DNA as we with the use of the oligo monitored the in-sample methyl transferase efficiency.

### EXAMPLE 2

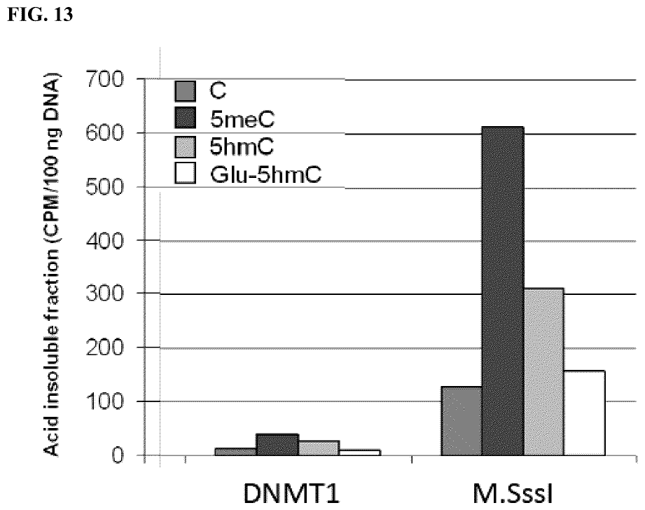

Addition of a chemical group to 5hmC, such as glucose can be performed to increase the ratio of methyl transferase efficiency between 5meC and 5hmC. See FIG. 13. Sterical blocking of the methyl transferase at the modified 5hmC position can be taken advantage of to increase the robustness of the methyl transferase dependent assay. Here we show the blocking effect of the addition of a glucose to 5hmC in a radioactive methyl transferase assay. Both DNMT1 and M.SssI can be efficiently blocked by the addition of a chemical group with a size larger than what can fit into the methyl transferase pocket, for example by the addition of glucose. By logical reasoning from our data and the previous demonstration of that a cytosine carbon-5 group of —CCCH3 (size of 6.1 Å) does not fit into the methyl transferase pocket (Valinkluck and Sovers, Cancer Res, 2007) one can assume that the addition of any chemical group to 5hmC which makes the total group too large for the methyl transferase pocket will be useful to increase the ratio of methyl transferase efficiency between 5meC and 5hmC.

### REFERENCES

1. Penn, N. W. Modification of brain deoxyribonucleic acid base content with maturation in normal and malnourished rats. Biochem J 155, 709-712 (1976).
2. Cannon-Carlson, S. V., Gokhale, H. & Teebor, G. W. Purification and characterization of 5-hydroxymethyluracil-DNA glycosylase from calf thymus. Its possible role in the maintenance of methylated cytosine residues. J Biol Chem 264, 13306-13312 (1989).
3. Tahiliani, M. et al. Conversion of 5-methylcytosine to 5-hydroxymethylcytosine in mammalian DNA by MLL partner TET1. Science (New York, N.Y 324, 930-935 (2009).
4. Kriaucionis, S. & Heintz, N. The nuclear DNA base 5-hydroxymethylcytosine is present in Purkinje neurons and the brain. Science (New York, N.Y 324, 929-930 (2009).
5. Ito, S. et al. Role of Tet proteins in 5mC to 5hmC conversion, ES-cell self-renewal and inner cell mass specification. Nature 466, 1129-1133 (2010).
6. Szwagierczak, A., Bultmann, S., Schmidt, C. S., Spada, F. & Leonhardt, H. Sensitive enzymatic quantification of 5-hydroxymethylcytosine in genomic DNA. Nucleic acids research 38, e181 (2010).
7. Ko, M. et al. Impaired hydroxylation of 5-methylcytosine in myeloid cancers with mutant TET2. Nature 468, 839-843 (2010).
8. Guo, J. U., Su, Y., Zhong, C., Ming, G. L. & Song, H. Hydroxylation of 5-methylcytosine by TET1 promotes active DNA demethylation in the adult brain. Cell 145, 423-434 (2011).
9. Wu, H. et al. Genome-wide analysis of 5-hydroxymethylcytosine distribution reveals its dual function in transcriptional regulation in mouse embryonic stem cells. Genes & development 25, 679-684 (2011).
10. Wu, H. et al. Dual functions of Tet1 in transcriptional regulation in mouse embryonic stem cells. Nature (2011).
11. Robertson, J., Robertson, A. B. & Klungland, A. The Presence of 5-hydroxymethylcytosine at the gene promotor and not in the gene body negatively regulates gene expression. Biochem Biophys Res Comm (2011).
12. Robertson, A. B. et al. A novel method for the efficient and selective identification of 5-hydroxymethylcytosine in genomic DNA. Nucleic acids research 39, e55 (2011).
13. Georgopoulos, C. P. & Revel, H. R. Studies with glucosyl transferase mutants of the T-even bacteriophages. Virology 44, 271-285 (1971).
14. Kornberg, S. R., Zimmerman, S. B. & Kornberg, A. Glucosylation of deoxyribonucleic acid by enzymes from bacteriophage-infected Escherichia coli. J Biol Chem 236, 1487-1493 (1961).
15. Gommers-Ampt, J. H. et al. beta-D-glucosyl-hydroxymethyluracil: a novel modified base present in the DNA of the parasitic protozoan T. brucei. Cell 75, 1129-1136 (1993).
16. Borst, P. & Sabatini, R. Base J: discovery, biosynthesis, and possible functions. Annu Rev Microbiol 62, 235-251 (2008).
17. van Leeuwen, F. et al. beta-D-glucosyl-hydroxymethyluracil is a conserved DNA modification in kinetoplastid protozoans and is abundant in their telomeres. Proc Natl Acad Sci USA 95, 2366-2371 (1998).
18. Sabatini, R., Meeuwenoord, N., van Boom, J. H. & Borst, P. Recognition of base J in duplex DNA by J-binding protein. J Biol Chem 277, 958-966 (2002).
19. Cross, M. et al. The modified base J is the target for a novel DNA-binding protein in kinetoplastid protozoans. EMBO J 18, 6573-6581 (1999).
20. Grover, R. K. et al. O-glycoside orientation is an essential aspect of base J recognition by the kinetoplastid DNA-binding protein JBP1. Angewandte Chemie (International ed 46, 2839-2843 (2007).
21. Ficz, G. et al. Dynamic regulation of 5-hydroxymethylcytosine in mouse ES cells and during differentiation. Nature (2011).
22. Stroud, H., Feng, S., Morey Kinney, S., Pradhan, S. & Jacobsen, S. E. 5-hydroxymethylcytosine is associated with enhancers and gene bodies in human embryonic stem cells. Genome Biol 12, R54 (2011).
23. Pastor, W. A. et al. Genome-wide mapping of 5-hydroxymethylcytosine in embryonic stem cells. Nature 473, 394-397 (2011).
24. Flusberg, B. A. et al. Direct detection of DNA methylation during single-molecule, real-time sequencing. Nat Methods 7, 461-465 (2010).
25. Song, C. X., Yu, M., Dai, Q. & He, C. Detection of 5-hydroxymethylcytosine in a combined glycosylation restriction analysis (CGRA) using restriction enzyme Taq(alpha)I. Bioorg Med Chem Lett (2011).
26. Xu, S. Y., Corvaglia, A. R., Chan, S. H., Zheng, Y. & Linder, P. A type IV modification-dependent restriction enzyme SauUSI from Staphylococcus aureus subsp. aureus USA300. Nucleic acids research (2011).
27. Szwagierczak, A. et al. Characterization of PvuRts1I endonuclease as a tool to investigate genomic 5-hydroxymethylcytosine. Nucleic acids research (2011).
28. Song, C. X. et al. Selective chemical labeling reveals the genome-wide distribution of 5-hydroxymethylcytosine. Nature biotechnology 29, 68-72 (2011).
29. Ficz, G. et al. Dynamic regulation of 5-hydroxymethylcytosine in mouse ES cells and during differentiation.Nature 473, 398-402 (2011).
30. Nestor, C., Ruzov, A., Meehan, R. & Dunican, D. Enzymatic approaches and bisulfite sequencing cannot distinguish between 5-methylcytosine and 5-hydroxymethylcytosine in DNA. Biotechniques 48, 317-319 (2010).
31. Studier, F. W. Protein production by auto-induction in high density shaking cultures. Protein Expr Purif 41, 207-234 (2005).
32. Zhang, Y. et al. Model-based analysis of ChIP-Seq (MACS). Genome Biol 9, R137 (2008).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the medical, biological and chemical sciences are intended to be within the scope of the following claims.

We claim:

1. A process for detecting 5-methylated and/or other modified cytosine residues in a nucleic acid sample comprising:
a) replicating said nucleic acid sample under conditions such that 5-methylated cytosine residues are maintained and said other modified cytosine residues are diluted;
b) treating said replicated nucleic acid sample to convert unmodified cytosine residues to a uracil or thymidine residues; and
c) reading the sequence of said replicated nucleic acid sample wherein 5-hydroxymethylated cytosine residues are identified as residues that are read by sequencing as a thymidine or uracil residue in said replicated nucleic acid sample.

2. The process of claim 1, wherein said nucleic acid sample is divided into at least first and second portions and said replicating and treating steps are performed on said first portion, and comparing the sequence of said first nucleic acid portion with the sequence of said second nucleic acid portion, wherein said other modified cytosine residues are identified as residues that are read by sequencing as a uracil or thymidine residue in said first nucleic acid portion and as a cytosine residue at the corresponding position in said second nucleic acid portion and wherein 5-methylated cytosine residues are identified as residues that are read as cytosine residues in both of said first and second nucleic acid portions.

3. The process of claim 2, wherein said treating said first and second portions to convert unmodified cytosine residues to thymidine residues further comprises treating said first and second nucleic acid portions with bisulfite to convert unmodified cytosine residues to uracil resides and replicating said first and second nucleic acid portions with a polymerase to convert said uracil residues into thymidine residues.

4. The process of claim 2, wherein said replicating of said first portion further comprises:
a) replicating said nucleic acid with a tagged primer to provide tagged replicated nucleic acid;
b) treating said tagged replicated nucleic acid strands with a DNA methyltransferase to provide tagged 5-methylcytosine-modified replicated nucleic acid;
c) isolating said tagged 5-methylcytosine-modified replicated nucleic acid;
d) treating said isolated tagged 5-methylcytosine-modified replicated nucleic acid with bisulfite to convert unmodified cytosine residues to uracil residues; and
e) replicating said isolated tagged bisulfite-treated nucleic acid with a polymerase to provide a first bisulfite treated nucleic acid portion.

5. The process of claim 4, wherein said tagged primer is a biotinylated primer.

6. The process of claim 1, wherein said other modified cytosine residues are selected from the group consisting of 5-hydroxymethyl cytosine, b-glu-5-hydroxymethyl cytosine, 5-formyl-cytosine and 5-carboxycytosine.

7. The process of claim 1, wherein said replicating said first portion under conditions such that 5-methylated cytosine residues are maintained and 5-hydroxymethylated cytosine residues are diluted comprises replicating said nucleic acid with a polymerase to provide replicated nucleic acid and treating said replicated nucleic acid with an enzyme to 5-methylate cytosine residues.

8. The process of claim 1, wherein said steps of replication and treating with an enzyme are performed one or more times.

9. The process of claim 1, wherein said steps of replication and treating with an enzyme are repeated 5 or more times.

10. The process of claim 1, wherein said steps of replication and treating with an enzyme are repeated 7 or more times.

11. The process of claim 1, wherein said steps of replication and treating with an enzyme are repeated 10 or more times.

12. The process of claim 1, wherein said steps of replication and treating with an enzyme are performed from about 1 to about 20 times or more.

13. The process of claim 1, wherein said replication is by a polymerase chain reaction.

14. The process of claim 1, wherein said replication is by a primer extension reaction.

15. The process of claim 1, wherein said replication utilizes a biotinylated primer.

16. The process of claim 1, wherein said enzyme is a DNA methyltransferase.

17. The process of claim 16, wherein said DNA methyltransferase is selected from the group consisting of mouse DNMT1, human DNMT1 and M.SssI DNMT.

18. The process of claim 1 further comprising the step of modifying 5-hydroxymethylated cytosine residues in said samples to prevent methylation of said 5-hydroxymethylated cytosine residues.

19. The process of claim 18, wherein said 5-hydroxymethylated cytosine residues are modified by addition of a blocking group.

20. The process of claim 19, wherein said blocking group is selected from the group consisting of beta-glucose, alpha-glucose); 6-O-β-D-glucopyranosyl-D-glucose, 6-O-alpha-D-glucopyranosyl-D-glucose; keto-glucose; azide-glucose; and modified versions thereof.

21. The process of claim 1, wherein said nucleic acid sample is selected from the group consisting of human, plant, mouse, rabbit, hamster, primate, fish, bird, cow, sheep, pig, viral, bacterial and fungal nucleic acid samples.

22. The process of claim 1, further comprising comparing the presence of 5-hydroxymethylcytosine and/or 5-methylcytosine in said nucleic acid in said sample to a reference standard, wherein an increased or decreased level of 5-hydroxymethylcytosine and/or 5-methylcytosine in said nucleic acid is indicative of the presence of a disease or of the probable course of a disease.

23. The process of claim 1, further comprising the step of providing a diagnoses or prognoses based on an increased or decreased level of 5-hydroxymethylcytosine and/or 5-methylcytosine in said nucleic acid as compared to a reference standard.

24. The process of claim 23, wherein said disease is cancer.

25. The process of claim 1, wherein said nucleic acid sample is genomic DNA.

26. A process for detecting methylated and hydroxymethylated cytosine residues in a nucleic acid sample comprising:
a) dividing said sample into at least first and second untreated portions;
b) replicating said first portion with a tagged primer and a polymerase to provide parent and tagged replicated nucleic acid;
c) treating said parent and said tagged replicated nucleic acid strands with a DNA methyltransferase to provide tagged 5-methylcytosine-modified replicated nucleic acid;
d) isolating said tagged 5-methylcytosine-modified replicated nucleic acid;
e) treating said isolated tagged 5-methylcytosine-modified replicated nucleic acid with bisulfite to convert unmodified cytosine residues to uracil residues;
f) replicating said isolated tagged bisulfite-treated nucleic acid with a polymerase to provide a first bisulfite treated nucleic acid portion;
g) sequencing said first bisulfite treated nucleic acid portion;
h) treating said second portion with bisulfite to convert unmodified cytosine residues to uracil residues;
i) replicating said bisulfite-treated nucleic acid with a polymerase to provide a second bisulfite treated nucleic acid portion;
j) sequencing said second bisulfite treated nucleic acid portion; and
k) comparing the sequence of said first bisulfite treated nucleic acid portion with the sequence of said second bisulfite treated portion, wherein 5-hydroxymethylated cytosine residues are identified as residues that are read by sequencing as a uracil or thymidine residue in said first bisulfite treated nucleic acid portion and as a cytosine residue at the corresponding position in said second bisulfite treated nucleic acid portion and wherein 5-methylated cytosine residues are identified as residues that are read as cytosine residues in said first and second bisulfite treated portions.

27. A process for predicting a predisposition to a disease in a subject, diagnosing a disease in a subject, predicting the likelihood of recurrence of disease in a subject, providing a prognosis for a subject with a disease, or selecting a subject with a disease for treatment with a particular therapy, comprising:
a) providing a genomic DNA sample from said subject; and
b) detecting the methylation status of predetermined portions of said genomic DNA sample by the process of claim 1,
wherein an altered level of 5-hydroxymethylcytosine and/or 5-methylcytosine methylation of said predetermined portions of said genomic DNA to a reference methylation status provides an indication selected from the group consisting of an indication of a predisposition of the subject to a disease, an indication that the subject has a disease, an indication of the likelihood of recurrence of a disease in the subject, an indication of survival of the subject, and an indication that the subject is a candidate for treatment with a particular therapy.

28. The process of claim 27, wherein said disease is a cancer.

29. The process of claim 27, wherein said subject is a human.
